(19) United States
(12) Patent Application Publication
(10) Pub. No.: US 2014/0186837 A1
(43) Pub. Date: Jul. 3, 2014

(54) Methods For Diagnosing Cancer

(71) Applicants: THE NATIONAL INSTITUTE FOR BIOTECHNOLOGY IN THE NEGEV LTD., Beer Sheva (IL); BEN-GURION UNIVERSITY OF THE NEGEV RESEARCH AND DEVELOPMENT AUTHORITY, Beer Sheva (IL)

(72) Inventor: Varda Shoshan-Barmatz, Omer (IL)

(73) Assignees: THE NATIONAL INSTITUTE FOR BIOTECHNOLOGY IN THE NEGEV LTD., Beer Sheva (IL); BEN-GURION UNIVERSITY OF THE NEGEV RESEARCH AND DEVELOPMENT AUTHORITY, Beer Sheva (IL)

(21) Appl. No.: 14/201,262

(22) Filed: Mar. 7, 2014

Related U.S. Application Data
(63) Continuation of application No. PCT/IL2012/050347, filed on Sep. 6, 2012.
(60) Provisional application No. 61/532,150, filed on Sep. 8, 2011.

(51) Int. Cl. C12Q 1/68; G01N 33/574

(52) U.S. Cl. CPC C12Q 1/6886; G01N 33/574
USPC 435/6.11; 435/7.1

(57) ABSTRACT

Methods and kits for the detection of cancer and for pre-cancer screening based on the expression of genes associated with altered metabolism and apoptosis in cancerous cells, particularly the expression of a mitochondrial antiviral-signaling (MAVS) or a voltage-dependent anion channel 1 (VDAC1) protein or mRNA in combination with additional genes associated with cell metabolism and/or apoptosis.

### FIELD OF THE INVENTION

The present invention relates to methods and kits for the detection of cancer and for pre-cancer screening based on the expression of genes associated with altered metabolism and apoptosis in cancerous cells, particularly the expression of a mitochondrial antiviral-signaling (MAVS) and/or a voltage-dependent anion channel 1 (VDAC1) protein or mRNA in combination with additional genes associated with cell metabolism and/or apoptosis.

### BACKGROUND OF THE INVENTION

An apoptotic program is present in latent form in all cell types throughout the body. Apoptosis can be activated through the extrinsic pathway, mediated by death receptors (e.g., CD-95 and TNF-R) that are activated by signals such toxins, hormones, growth factors, nitric oxide or cytokines, and through the mitochondria-mediated, intrinsic pathway. The intrinsic pathway is initiated in response to different stimuli, including high levels of cytoplasmic Ca²⁺, reactive oxygen species (ROS), activation of pro-apoptotic Bcl-2 family proteins, and UV damage. These stimuli provoke the outer mitochondrial membrane (OMM) permeabilization leading to the release of pro-apoptotic proteins from the intermembrane space to the cytosol, leading to caspases activation and subsequently to cell death. The pro-apoptotic proteins include including Cytochrome c (Cyto c), second mitochondria-derived activator of caspases (Smac/DIABLO), and apoptosis inducing factor (AIF). Defects in the regulation of apoptosis are often associated with disease and drug resistance, with evading apoptosis being a hallmark of cancer (Hanahan, D. and R. A. Weinberg, Hallmarks of cancer: the next generation. Cell, 2011. 144(5), 646-74; Shoshan-Barmatz, V. and Golan, M. Mitochondrial VDAC1: function in cell life and death and a target for cancer therapy. Curr Med Chem, 2012. 19(5), 714-35). Cancer cells utilize a variety of strategies to limit or circumvent apoptosis including quenching of the mitochondrial apoptotic pathway by over-expression of anti-apoptotic proteins such as the Bcl-2 family proteins and hexokinases (HK), preventing the release of Cytochrome c from the mitochondria and thereby preventing apoptotic cell death.

In recent years, a body of evidence has accumulated indicating a correlation between alterations in metabolism and cancer formation. It is well known that cancer cells undergo significant metabolic adaptation (Wenger, J. B. et al. Combination therapy targeting cancer metabolism. Med Hypotheses 2011, 76, (2), 169-172). Normal cells produce energy by low rate glycolysis followed by oxidation of pyruvate in mitochondria, whereas malignant cancer cells typically display high rates of glycolysis even when fully oxygenated (Gatenby, R. A. and Gillies, R. J. Why do cancers have high aerobic glycolysis? Nat Rev Cancer 2004, 4, (11), 891-899) and are subject to suppressed mitochondrial respiration, despite the fact that glycolysis is a less energy-efficient pathway (Kim, J. W. and Dang, C. V. Cancer's molecular sweet tooth and the Warburg effect. Cancer Res 2006, 66, (18), 8927-8930). This phenomenon is known as the Warburg effect (Warburg, O. On the origin of cancer cells. Science 1956, 123, (3191), 309-314). Indeed, in aerobic tissues, glycolysis contributes only 5% of cellular ATP production, while in tumor cells glycolysis account for 50% to 70% of ATP formation which is advantageous for rapid growing tumors (Mathupala, S. P. et al. Hexokinase-2 bound to mitochondria: cancer's stygian link to the “Warburg Effect” and a pivotal target for effective therapy. Semin Cancer Biol2009, 19, (1), 17-24).

One of the mitochondrial proteins controlling cell life and death is the voltage-dependent anion channel (VDAC), also known as mitochondrial porin. VDAC is a pore-forming protein found in the outer mitochondrial membrane in all eukaryotic cells controlling the fluxes of ions and metabolites between the mitochondria and the cytosol. The VDAC protein has a key role in cell energy metabolism and in the regulation of apoptosis, and serves as a control gate defining whether the mitochondria function normally in respiration or whether the mitochondrial metabolism is suppressed, leading to apoptotic cell death. Thus, VDAC appears to act as a convergence point for a variety of cell survival and cell death signals, mediated by its association with various ligands and proteins.

Several studies have demonstrated remarkable differences in the expression levels of VDACs between cancer cells lines and non-cancerous cells. The transcript levels of the three VDAC isoforms in rat liver hepatoma cell line (AH130) are significantly higher than those in normal liver (Shinohara, Y. et al. Characterization of porin isoforms expressed in tumor cells. Eur J Biochem 2000, 267, (19), 6067-6073). When the expression levels of VDAC1 in human cancer cell lines and normal cell lines were compared, it was shown that cancer cells expressed higher VDAC1 levels than do normal WI-38 fibroblast cells (Simamura, E. et al. Furanonaphthoquinones cause apoptosis of cancer cells by inducing the production of reactive oxygen species by the mitochondrial voltage-dependent anion channel. Cancer Biol Ther 2006, 5, (11), 1523-1529). High expression levels of VDAC1 were also found in non-small cell lung cancer cell line (NSCLC) (Grills, C. et al. Gene expression meta-analysis identifies VDAC1 as a predictor of poor outcome in early stage non-small cell lung cancer. PLoS One 2011, 6, (1), e14635). This study identified VDAC1 gene expression as a predictor of poor outcome in NSCLC and other cancers and showed association of VDAC1 over-expression with dysregulation of a conserved set of biological pathways, which may be causally associated with aggressive tumor behavior. In a proteomic analysis of the aging-related proteins in human normal colon epithelial tissue, nineteen proteins, including VDAC1 and VDAC2, were significantly up-regulated (Li, M. et al. Proteomic analysis of the aging-related proteins in human normal colon epithelial tissue. J Biochem Mol Biol 2007, 40, (1), 72-81). VDAC expression level was changed significantly in colorectal cancer as analyzed using mass spectrometry and immunohistochemistry using tissue microarrays (Alfonso, P. et al. Proteome Analysis of Membrane Fractions in Colorectal Carcinomas by using 2D-DIGE Saturation Labeling, Journal of Proteome Research 2008, 7, 4247-4255).

U.S. Patent Application Publication No. 2004/0171003 discloses large number of cancer-associated genes, among them the gene encoding VDAC1, and a method for detecting a cancer on the basis of expression of the cancer-associated genes. The application further discloses an array based on the cancer-associated genes, a kit for detecting a cancer and a method for suppressing proliferation of a cancer cell.

Host responses to viral infection include both immune activation and programmed cell death. The mitochondrial antiviral-signaling protein (MAVS), also known by the names VISA (virus-induced signaling adapter), IPS-1 and Cardif, is critical for host defenses to viral infection by the activation of NF-κB and IRF3 in response to viral infection leading to the induction of type-1 interferons (IFN-I). MAVS contains an N-terminal CARD-like domain and a C-terminal transmembrane domain, both of which are essential for MAVS signaling. The transmembrane domain targets MAVS to the mitochondria. Its role in viral-induced cell apoptosis has not been fully elucidated.

MAVS not only plays a pivotal role in the induction of anti-viral and inflammatory pathways but is also involved in the coordination of apoptotic and metabolic functions (Belgnaoui, S. M., S. Paz, and J. Hiscott, Orchestrating the interferon antiviral response through the mitochondrial antiviral signaling (MAVS) adapter. Curr Opin Immunol 2011, 23(5), 564-72) MAVS is active only when bound to mitochondria and is proposed to regulate cell death by various mechanisms (Seth, R. B., et al., Identification and characterization of MAVS, a mitochondrial antiviral signaling protein that activates NF-kappaB and IRF 3. Cell 2005. 122(5), 669-82; Lei, Y., et al., MAVS-mediated apoptosis and its inhibition by viral proteins. PLoS ONE 2009. 4(5), e5466) including via modulation of VDAC1 expression levels (Xu, Y., H. Zhong, and W. Shi, MAVS protects cells from apoptosis by negatively regulating VDAC1. Mol Cell Biochem 2010, e-publication)

Cancer is the second leading cause of death behind heart diseases. Despite recent advances, the challenge of cancer diagnosis and further treatment remains a timely diagnosis of the appearance of cancerous cells and targeted treatment regimens that are specific to the cancer type and, optimally, to the individual suffering from the cancer.

Thus, there is a recognized need for, and would be highly advantageous to have a therapeutic cellular index designed for diagnostic, prognostic and treatment guidance.

### SUMMARY OF THE INVENTION

The present invention provides means and methods for diagnosing and monitoring the progress of various types of cancers, including precancerous lesions, non-solid and solid tumors.

The present invention is based in part on the expression profile of genes and proteins involved in the metabolism re-programming of cancer cells and in their capability to evade apoptosis. The present invention discloses for the first time that the mitochondrial antiviral-signaling (MAVS) protein is overexpressed in cancer cells, including cancerous cells of non-solid as well as solid tumors. The expression is concomitant with the expression of the voltage-dependent ion channel isoform 1 (VDAC1). Unexpectedly, cancerous cells of non-solid tumors, particularly leukemia, also overexpress the mitochondrial pro-apoptotic proteins SMAC/DIABLO (second mitochondria-derived activator of caspases) and AIF (apoptosis-inducing factor). The AIF protein is also differentially expressed in breast cancer cells.

Thus, according to one aspect, the present invention provides a method for diagnosing cancer in a subject, the method comprising determining a level of a mitochondrial antiviral-signaling (MAVS) protein (GeneBank accession Nos. NP_{—}001193420.1 and NP_{—}065797.2) or mRNA in a sample obtained from the subject, wherein MAVS level above a predetermined threshold is indicative for the presence of cancer cells in the subject, thereby diagnosing a cancer in said subject.

According to additional aspect, the present invention provides a method for diagnosing cancer in a subject, the method comprising determining a level of voltage-dependent anion channel 1 (VDAC1) protein (GeneBank accession Nos. ABM87491.1, ABM84109.1 and AAH90042.1) or mRNA in a sample obtained from the subject, wherein VDAC1 level above a predetermined threshold is indicative for the presence of cancer in the subject, thereby diagnosing cancer in said subject.

According to further aspect, the present invention provides a method for diagnosing cancer in a subject, the method comprising determining in a sample obtained from the subject a level of voltage-dependent anion channel 1 (VDAC1) protein (GeneBank accession Nos. ABM87491.1, ABM84109.1 and AAH90042.1) or mRNA and a level of at least one additional cancer marker selected from the group consisting of:

mitochondrial antiviral-signaling (MAVS, GeneBank accession Nos. NP_{—}001193420.1 and NP_{—}065797.2) protein or mRNA;

second mitochondria-derived activator of caspases (SMAC/DIABLO, UniProt accession No. Q9NR28 protein or mRNA;

apoptosis inducing factor (AIF, UniProt accession No. 095831 protein or mRNA;

apoptosis regulator Bcl-2 (UniPort accession No. P10415.2) protein or mRNA; and

hexokinase-1 (HK-1, GeneBank accession No. AAC15862.1) protein or mRNA; and any combination thereof,

wherein level above a predetermined threshold of the VDAC1 protein or mRNA and of the at least one additional protein or mRNA is indicative for the presence of cancer cells in the subject, thereby diagnosing cancer in said subject. Each possibility represents a separate embodiment of the resent invention.

According to certain embodiments, the method comprises determining the level of VDAC1 and MAVS, wherein level above a predetermined threshold of VDAC1 and MAVS protein or mRNA is indicative for the presence of cancer cells in the subject, thereby diagnosing cancer in the subject.

According to other embodiments, the method comprises determining the level of VDAC1 and SMAC/DIABLO. According to yet additional embodiments, the method comprises determining the level of VDAC1 and AIF.

According to further embodiments, the method comprises determining the level of VDAC1, HK-1 and at least one additional protein or mRNA selected from the group consisting of MAVS, SMAC/DIABLO and AIF. Each possibility represents a different embodiment of the present invention.

According to yet further embodiments, the method comprises determining the level of VDAC1, Bcl-2 and at least one additional protein or mRNA selected from the group consisting of MAVS, SMAC/DIABLO and AIF. Each possibility represents a different embodiment of the present invention.

According to additional embodiments, the method comprises determining the level of VDAC1, MAVS, SMAC/DIABLO and AIF. According to yet additional embodiments, the method comprises determining the level of VDAC1, MAVS, SMAC/DIABLO, AIF, Bcl-2 and HK-1.

According to additional aspect, the present invention provides a method for monitoring tumor progression in a subject, comprising the step of determining a level of voltage-dependent anion channel 1 (VDAC1) protein (GeneBank accession Nos. ABM87491.1, ABM84109.1 and AAH90042.1) or mRNA in a sample comprising tumor cells isolated from the subject, wherein VDAC1 level above a predetermined threshold correlates with tumor progression, thereby monitoring tumor progression. In some embodiments, VDAC1 level above a predetermined threshold positively correlates with tumor progression.

According to certain embodiments, the method further comprises determining in the sample the level of at least one additional protein or mRNA selected from the group consisting of Mitochondrial antiviral-signaling (MAVS, GeneBank accession Nos. NP_{—}001193420.1 and NP_{—}065797.2); Second mitochondria-derived activator of caspases (SMAC/DIABLO, UniProt accession No. Q9NR28; Apoptosis inducing factor (AIF, UniProt accession No. 095831); Bcl-2 (UniPort accession No. P10415.2); Hexokinase-1 (HK-1, GeneBank accession No. AAC15862.1); and any combination thereof, wherein level above a predetermined threshold of VDAC1 and the at least one additional protein or mRNA correlates with tumor progression, thereby monitoring tumor progression.

According to a further aspect, the present invention provides a method for monitoring tumor progression in a subject, comprising the step of determining a level of Mitochondrial antiviral-signaling (MAVS) protein GeneBank accession Nos. NP_{—}001193420.1 and NP_{—}065797.2) or mRNA in a sample comprising tumor cells isolated from the subject, wherein MAVS level above a predetermined threshold correlates with tumor progression, thereby monitoring tumor progression. In some embodiments, MAVS level above a predetermined threshold positively correlates with tumor progression.

According to certain embodiments, the methods of the present invention are useful for the diagnosis or prognosis of non-solid tumors. According to these embodiments, the sample is selected from the group consisting of peripheral blood, lymph fluid and bone marrow. According to certain embodiments, the method comprises determining the level of a protein. According to some embodiments, the peripheral blood sample comprises cells. According to some embodiments, the peripheral blood cells are nucleated cells.

According to certain typical embodiments, the non-solid tumor is leukemia.

According to other embodiments, the methods of the present invention are useful for the diagnosis or prognosis of solid tumors. According to these embodiments, the sample is a tissue sample or cells derived thereof.

According to certain embodiments, the methods of the present invention are particularly useful for the diagnosis or prognosis of a cancer selected from the group consisting of melanoma, thyroidal cancer, non-small lung cancer, prostate cancer, brain cancer, ovary cancer, pancreas cancer, cervix cancer, breast cancer and esophagus cancer. Each possibility represents a separate embodiment of the present invention.

According to a further aspect, the present invention provides a kit for the diagnosis or prognosis of cancer, the kit comprising: (i) at least two probes for detecting a voltage-dependent anion channel 1 (VDAC1) protein or mRNA; and at least one an additional cancer cell marker selected from the group consisting of mitochondrial antiviral-signaling (MAVS, GeneBank accession Nos. NP_{—}001193420.1 and NP_{—}065797.2) protein or mRNA; second mitochondria-derived activator of caspases (SMAC/DIABLO, UniProt accession No. Q9NR28) protein or mRNA; apoptosis inducing factor (AIF, UniProt accession No. 095831) protein or mRNA; Bcl-2 (UniPort accession No. P10415.2) protein or mRNA; hexokinase-1 (HK-1, GeneBank accession No. AAC15862.1) protein or mRNA; or any combination thereof; and (ii) a package insert comprising instructions for measuring the level of the VDAC1 protein or mRNA and the at least one additional cancer cell marker protein or mRNA.

According to certain embodiments, the kit comprises a nucleotide probe or primer capable of selectively hybridizing to VDAC1 mRNA and at least one additional probe or primer capable of selectively hybridizing to a mRNA of at least one of MAVS, SMAC/BIABLO, AIF, Bcl-2 and HK-1. According to some embodiments, the kit further comprises reagents for employing nucleotide-based technology (NAT)-based assay.

According to other embodiments, the kit comprises an antibody capable of selectively recognizing or interacting with VDAC1 protein and at least one additional antibody capable of selectively recognizing or interacting with at least one protein selected from the group consisting of MAVS, SMAC/DIABLO, AIF, Bcl-2 and HK-1. According to some embodiments, the kit further comprises reagents for performing an immunoassay.

Other objects, features and advantages of the present invention will become clear from the following description and drawings.

### BRIEF DESCRIPTION OF THE FIGURES

FIG. 1 shows representative blot of Western blot analysis of cell lysates of peripheral blood mononuclear cells (PBMCs) derived from chronic lymphocytic leukemia (CLL) patients and healthy donors probed with antibodies directed against MAVS [n=28 (CLL), 20 (Healthy)] and β-Actin.

FIG. 2 shows representative blot of Western blot analysis of cell lysates of PBMCs derived from CLL patients and healthy donors probed with antibodies directed against VDAC1 [n=28 (CLL), 20 (Healthy)] and β-Actin.

FIGS. 3A-B shows FACS analysis of CD19+ and CD5+ labeled PBMCs isolated from a healthy donor (FIG. 3A) or CLL individual (FIG. 3B). Percentage of CD19+/CD5+ cells was determined using monoclonal antibodies directed to CD19, a B-lymphocyte marker, and CD5, a T-lymphocyte marker, followed by FACS analysis. FIG. 3C shows VDAC1 expression levels as a function of the percentage of CD19+/CD5+ cells for each patient (R²=0.7). VDAC1 levels were assayed as in FIG. 2.

FIG. 4 shows representative blot of Western blot analysis of cell lysates of PBMCs derived from CLL patients and healthy donors probed with antibodies directed against AIF [n=28 (CLL), 20 (Helathy)] and β-Actin.

FIG. 5 shows representative blot of Western blot analysis of cell lysates of PBMCs derived from CLL patients and healthy donors probed with antibodies directed against SMAC/DIABLO [n=20 (CLL), 15 (Healthy)] and β-Actin.

FIG. 6 shows representative blot of Western blot analysis of cell lysates of PBMCs derived from CLL patients and healthy donors probed with antibodies directed against HK-1 [n=28 (CLL), 19 (Healthy)] and β-Actin.

FIG. 7 shows representative blot of Western blot analysis of cell lysates of PBMCs derived from CLL patients and healthy donors probed with antibodies directed against Bcl-2 [n=28 (CLL), 20 (Healthy)] and β-Actin.

FIG. 8 shows representative blot of Western blot analysis of cell lysates of PBMCs derived from CLL patients and healthy donors probed with antibodies directed against BAX [n=6 (P), 6 (H)] and β-Actin.

FIG. 9 is a bar graph showing a quantitative analysis of the expression profile (level) of MAVS, VDAC1, AIF, SMAC/DIABLO, HK-1 and Bcl-2 in PBMCs isolated from healthy donors and patients afflicted with CLL based on the blots presented in FIGS. 1-2 and 4-8 and additional samples. A difference between healthy and CLL groups was considered statistically significant when P<0.001 (***), P<0.01 (**), as determined by the Mann-Whitney test.

FIGS. 10A-E show a scatter plot presentation of the individual quantitative measures of the expression profile (level) of VDAC1 (FIG. 10A) MAVS (FIG. 10B), Bcl-2 (FIG. 10C) SMAC/DIABLO (FIG. 10D) and AIF (FIG. 10E) in PBMCs isolated from healthy donors and patients afflicted with CLL.

FIGS. 11A-D show correlation between the relative expression of VDAC1 and the relative expression of the apoptosis-related proteins, SMAC (FIG. 11A), AIF (FIG. 11B), Bcl-2 FIG. 11C) and MAVS (FIG. 11D) in CLL patients (open circles) and healthy donors (dark circles) as determined by linear regression. Values were calculated as in FIG. 9.

FIGS. 12A-H show a bi-variance analysis performed based on the relative expression of apoptosis-related proteins, considered as independent variables: VDAC1 (FIG. 12A), SMAC/Diablo (FIG. 12B), MAVS (FIG. 12C), AIF (FIG. 12D), HK-1 (FIG. 12E), HK-1+AIF (FIG. 12F), Bcl-2 (FIG. 12G) and Bax (FIG. 12H). Relative protein expression levels were those presented in FIG. 11. The dependents were determined as 0 for healthy donors and 100 for CLL patients. The binary logistic regression model was carried out with a 95% confidence interval.

FIG. 13 is a photograph of a gel, showing that VDAC1 protein levels are increased upon cancer transformation. A cell-based transformation model of primary human embryonic lung fibroblasts (WI-38) was employed to analyze the expression of VDAC1 protein. Cell lysates of the different cell lines, representing the various in vivo tumor progression stages are presented. The proteins were separated on a SDS-PAGE and identified by Western-Blot analysis. RU: relative units of VDAC1 expression relative to β-actin levels, as determined by densitometry analysis. Primary: embryonic lung primary fibroblasts. hTERT: hTERT-induced immortalization. Slow: slow growing immortalized WI-38 cells. Fast: fast growing, spontaneously acquired der(X)t(X;17) locus silencing WI-38 cells. GSE56: Inactivation of wild-type p53, using the dominant-negative polypeptide GSE56. H-Ras: H-RasV12.

FIGS. 14A-B demonstrate the overexpression of VDAC1 and MAVS in various tumors using tissue microarrays from US-Biomax, including 30 different malignant cases and 7 cases of normal controls per cancer type. Broad range of tumor types was analyzed, including cervix, lung, thyroid, melanoma and brain cancer (FIG. 14A) and pancreas, prostate liver and bladder (FIG. 14B). Formalin-fixed and Paraffin-embedded tissue microarray slides were immunohistochemistry stained using specific anti VDAC or anti-MAVS antibodies with horse radish peroxidase (HRP) as a secondary antibody with diaminobenzidine (DAB) as the substrate. Slides were also stained with hematoxylin-eosin. Representative sections from normal and cancer tissue are shown. The numbers in the top indicate the percentage of the cancer cases stained at the shown intensity. Less than 100% for normal tissue indicates that the other sections stained at a higher intensity. In case of cancer tissue, less than 100% indicates lower staining intensity for the rest of the sections but with intensity higher than in the normal tissue.

### DETAILED DESCRIPTION OF THE INVENTION

The present invention provides tools for the diagnosis and prognosis of non-solid and solid tumors, based on the differential expression of several proteins in cancer cells compared to healthy cells. The proteins are involved in the cell energy production and/or apoptosis. Without wishing to be bound by any specific theory or mechanism of action, the expression profile of these proteins may point to the survival strategy of cancer cells involving metabolic re-programming and anti-apoptotic defense mechanisms. The expression profile of these proteins can thus serve not only for cancer diagnosis and progression but also for the selection of treatment targeted to modulate the aberrant metabolic or apoptotic function of the cancer cells.

### DEFINITIONS

As used herein, the term “MAVS” refers to any isoform of mitochondrial antiviral-signaling protein and to polynucleotide encoding same. The protein is also named induced signaling adaptor (VISA), interferon-promoter stimulator 1 (IPS-1) and caspase recruitment domain (CARD) adaptor inducing interferon (CARDIF). MAVS protein has an important role in raising the anti-viral defense in the cell. MAVS is further defined herein by its GeneBank accession Nos. NP_{—}001193420.1 and NP_{—}065797.2.

The terms “VDAC” and “VDAC1” are used herein interchangeably and refer to isoform 1 of the voltage-dependent anion channel and to polynucleotide encoding same. VDAC is a pore-forming protein found in the outer mitochondrial membrane in all eukaryotic cells controlling the fluxes of ions and metabolites between the mitochondria and the cytosol. VDAC1 is the main isoform expressed in mammalian cells. VDAC1 is further defined herein by its GeneBank accession Nos. ABM87491.1, ABM84109.1 and AAH90042.1.

The terms “SMAC/DIABLO” and “SMAC” re used herein interchangeably and refer to second mitochondria-derived activator of caspases/direct inhibitor of apoptosis protein (IAP) binding protein with low pl. The term also refers to polynucleotides encoding this protein. SMAC is a mitochondrial protein that promotes cytochrome-c dependent activation by eliminating the inhibition via IAP—a protein that negatively regulates apoptosis or programmed cell death. SMAC/DIABLO is further defined herein by its UniProt accession No. Q9NR28.

The term “AIF” as used herein refers to apoptosis inducing factor as well as to polynucleotides encoding same. AIF is involved in initiating a caspase-independent pathway of apoptosis (positive intrinsic regulator of apoptosis) by causing DNA fragmentation and chromatin condensation. It also acts as an NADH oxidase. Another AIF function is to regulate the permeability of the mitochondrial membrane upon apoptosis. AIF is further defined herein by its UniProt accession No. 095831.

The term “Bcl-2” as used herein refers B-cell lymphoma-2 protein and to polynucleotides encoding same. Bcl-2 is the founding member of the Bcl-2 family of apoptosis regulating proteins, and is known to have an anti-apoptotic activity. Bcl-2 is further defined herein by its UniPort accession No. P10415.2.

HK-1 as used herein refers to Hexokionase-1 and polynucleotides encoding same. Hexokinases phosphorylate glucose to produce glucose-6-phosphate, thus committing glucose to the glycolytic pathway. Hexokinase-1 is a ubiquitous form of hexokinase which localizes to the outer membrane of mitochondria. HK-1 is further defined herein by its GeneBank accession No. AAC15862.1.

As used herein, the term “cancer cell marker” refers to proteins or polynucleotides of the present invention, the expression of which is indicative of the cell being a cancerous cell. A “cancer cell marker probe” is a detectable and/or labeled probe capable of interacting with a cancer cell marker.

The term “diagnosing” as used herein refers to determining presence or absence of pathology, classifying pathology or a symptom or determining a severity of the pathology. The term diagnosis also refers, in some embodiments, to screening. Screening for cancer, in some embodiments, can lead to earlier diagnosis in specific cases.

The term “prognosis” as used herein refers to forecasting an outcome of pathology and/or prospects of recovery including the efficacy of medication or treatment. In some embodiments, the term “prognosis” further refers to the determination of tumor progress.

According to one aspect, the present invention provides a method for diagnosing cancer in a subject, comprising determining a level of a mitochondrial antiviral-signaling (MAVS) protein (GeneBank accession Nos. NP_{—}001193420.1 and NP_{—}065797.2) or mRNA in a sample obtained from the subject, wherein MAVS level above a predetermined threshold is indicative for the presence of cancer cells in the subject, thereby diagnosing a cancer in said subject.

According to certain embodiments, the method is for diagnosing non-solid tumors. According to other embodiments, the method is for diagnosing solid tumors. According to yet additional embodiments, the method is for diagnosing precancerous lesions.

According to certain embodiments, the present invention provides a method for diagnosing a blood cancer in a subject, comprising determining a level of a mitochondrial antiviral-signaling (MAVS) protein (GeneBank accession Nos. NP_{—}001193420.1 and NP_{—}065797.2) or mRNA in a blood sample obtained from the subject, wherein MAVS level above a predetermined threshold is indicative for the presence of cancer cells in the blood sample, thereby diagnosing blood cancer in said subject.

According to some embodiment, the sample is a sample of peripheral blood. According to certain embodiments, the blood sample comprises nucleated cells.

According to additional aspect, the present invention provides a method for diagnosing cancer in a subject, comprising determining a level of voltage-dependent anion channel 1 (VDAC1) protein (GeneBank accession Nos. ABM87491.1, ABM84109.1 and AAH90042.1) or mRNA in a sample obtained from the subject, wherein VDAC1 level above a predetermined threshold is indicative for the presence cancer cells in the subject, thereby diagnosing cancer in said subject.

According to certain embodiments, the method is for diagnosing non-solid tumors. According to other embodiments, the method is for diagnosing solid tumors. According to yet additional embodiments, the method is for diagnosing precancerous lesions.

In other embodiments, the present invention provides a method for diagnosing a blood cancer in a subject, comprising determining a level of voltage-dependent anion channel 1 (VDAC1) protein or mRNA in a blood sample obtained from the subject, wherein VDAC1 level above a predetermined threshold is indicative for the presence of cancer cells in the blood sample, thereby diagnosing blood cancer in said subject.

According to some embodiment, the sample is a sample of peripheral blood. According to certain embodiments, the blood sample comprises nucleated cells.

It is to be explicitly understood that the protein or polynucleotide levels can be determined directly in the sample as obtained from the subject, or the sample may be processed before measurements are performed. According to some embodiments, the sample is selected from the group consisting of blood, lymph fluid and bone marrow. According to other embodiments, the sample is processed to be cell free.

The field of cancer biomarkers is constantly expanding, and biomarkers are targeted to the diagnosis, prognosis, and predicting and measuring treatment efficacy. Many markers are based on gene mutations; however, it has been recently reported that other changes in the cells, including increased or decreased protein expression level and/or variation in protein post-translational modifications are responsible for the appearance and propagation of cancerous cells.

The present invention now discloses that the expression level of several proteins associated with cell metabolism and/or apoptosis is altered in cells isolated from various types of cancers. These proteins can be used as markers for diagnosing cancer cells, as well as new targets for cancer therapy. Without wishing to be bound by any specific theory or mechanism of action, the involvement of each of the cancer markers of the present invention in the cell metabolism and/or apoptosis may reflect the cancer cell death-defense mechanism, providing a new approach for the development of anti-cancer drugs.

Thus, according to further aspect, the present invention provides a method for diagnosing cancer in a subject, comprising determining in a sample obtained from the subject a level of voltage-dependent anion channel 1 (VDAC1) protein (GeneBank accession Nos. ABM87491.1, ABM84109.1 and AAH90042.1) or mRNA and a level of at least one additional cancer marker selected from the group consisting of:

mitochondrial antiviral-signaling (MAVS, GeneBank accession Nos. NP_{—}001193420.1 and NP_{—}065797.2) protein or mRNA;

second mitochondria-derived activator of caspases (SMAC/DIABLO, UniProt accession No. Q9NR28 protein or mRNA;

apoptosis inducing factor (AIF, GeneBank UniProt accession No. 095831) protein or mRNA;

Bcl-2 (GeneBank accession No. UniPort accession No. P10415.2) protein or mRNA; and

hexokinase-1 (HK-1, GeneBank accession No. AAC15862.1) protein or mRNA; and any combination thereof,

wherein level above a predetermined threshold of the VDAC1 protein or mRNA and of the at least one additional protein or mRNA is indicative for the presence of cancer cells in the subject, thereby diagnosing cancer in said subject. Each possibility represents a separate embodiment of the present invention.

According to certain embodiments, the method comprises determining the level of VDAC1 and MAVS, wherein level above a predetermined threshold of VDAC1 and MAVS protein or mRNA is indicative for the presence of cancer cells in the subject, thereby diagnosing cancer in said subject.

According to other embodiments, the method comprises determining the level of VDAC1 and SMAC/DIABLO. According to yet additional embodiments, the method comprises determining the level of VDAC1 and AIF.

According to further embodiments, the method comprises determining the level of VDAC1, HK-1 and at least one additional protein or mRNA selected from the group consisting of MAVS, SMAC/DIABLO and AIF. Each possibility represents a different embodiment of the present invention.

According to yet further embodiments, the method comprises determining the level of VDAC1, Bcl-2 and at least one additional protein or mRNA selected from the group consisting of MAVS, SMAC/DIABLO and AIF. Each possibility represents a different embodiment of the present invention.

According to additional embodiments, the method comprises determining the level of VDAC1, MAVS, SMAC/DIABLO and AIF. According to yet additional embodiments, the method comprises determining the level of VDAC1, MAVS, SMAC/DIABLO, AIF, Bcl-2 and HK-1.

As is appreciate to the skilled artisan in view of the Example section presented hereinbelow, the methods of the present invention are useful for the diagnosis of non-solid as well as of solid tumors.

According to certain embodiments, a non-solid tumor is a blood cancer. In some embodiments, a non-solid tumor or blood cancer is leukemia or lymphoma. In other embodiments, a non-solid tumor or blood cancer is acute lymphoblastic leukemia (ALL). In some embodiment, a non-solid tumor or blood cancer is chronic lymphocytic leukemia (CLL). In other embodiments, a non-solid tumor or blood cancer is chronic myelogenous leukemia (CML). In yet other embodiments, a non-solid tumor or blood cancer is acute myelogenous leukemia (AML). In additional embodiments, a non-solid tumor or blood cancer is small lymphocytic lymphoma (SLL). In yet additional embodiments, a non-solid tumor or blood cancer is acute monocytic leukemia (AMOL). In further embodiments, a non-solid tumor or blood cancer is Hodgkin's lymphomas (any of the four subtypes). In some embodiments, a non-solid tumor or blood cancer is Non-Hodgkin's lymphomas (any of the subtypes). In additional embodiments, a non-solid tumor or blood cancer is myeloid leukemia.

Methods for obtaining a blood or tissue samples are known to a person skilled in the art.

In other embodiments, the level of VDAC1 protein or mRNA in a cell correlates with tumor progression. In another embodiment, higher level of VDAC1 protein or mRNA in a cell correlates with later stages of tumor progression. In another embodiment, provided herein is a method for determining tumor progression, comprising the step of determining a level of VDAC1 protein or mRNA in a sample comprising tumor cells, wherein VDAC1 protein or mRNA level above a predetermined threshold correlates with tumor progression.

In yet other embodiments, the level of MAVS protein or mRNA in a cell correlates with tumor progression. In another embodiment, higher level of MAVS protein or mRNA in a cell correlates with later stages of tumor progression. In another embodiment, provided herein is a method for determining tumor progression, comprising the step of determining a level of MAVS protein or mRNA in a sample comprising tumor cells, wherein MAVS protein or mRNA level above a predetermined threshold correlates with tumor progression.

In additional embodiments, a method for determining tumor progression includes a method for checking how a patient is responding to a treatment. In another embodiment, a method for determining tumor progression includes a method for monitoring cancer regression. In another embodiment, a method for determining tumor progression includes a method for monitoring cancer recurrence. In another embodiment, a decrease or return to a normal level of, for example, VDAC1 and/or MAVS may indicate that the cancer is responding to therapy, whereas an increase in the level of VDAC1 and/or MAVS may indicate that the cancer is not responding to therapy.

In other embodiments, a method for determining the presence of a tumor or tumor progression comprises measuring the level of VDAC1 and/or MAVS and at least one additional cancer cell marker (protein or mRNA) selected from the group comprising: HK-1, Bcl-2, SMAC/DIABLO, AIF or any combination thereof.

In other embodiments, a kit of the invention comprises: a first probe for detecting a voltage-dependent anion channel 1 (VDAC1) protein or mRNA, a second probe for detecting at least one additional cancer cell marker, and a package insert comprising instructions for measuring the level of: (a) VDAC1 protein or mRNA; and (b) at least one additional cancer cell marker.

According to certain embodiments, the at least one additional cancer cell marker is selected from the group consisting of mitochondrial antiviral-signaling (MAVS, GeneBank accession Nos. NP_{—}001193420.1 and NP_{—}065797.2) protein or mRNA; second mitochondria-derived activator of caspases (SMAC/DIABLO, UniProt accession No. Q9NR28) protein or mRNA; apoptosis inducing factor (AIF, UniProt accession No. 095831) protein or mRNA; Bcl-2 (UniPort accession No. P10415.2) protein or mRNA; and hexokinase-1 (HK-1, GeneBank accession No. AAC15862.1) protein or mRNA.

According to additional embodiments, the at least one additional cancer cell marker is selected from the group consisting of: mitochondrial antiviral-signaling (MAVS) protein or mRNA, p53, Rb, EGFR, hexokinase antibody, Alpha fetoprotein (AFP), HK-1, HK-II, Bcl-2, Bax, CA15-3, CA19-9, Calretinin, a carcinoembryonic antigen, CD34, CD99, CD117, Chromogranin, Cytokeratin (various types), Desmin, Epithelial membrane protein (EMA), Factor VIII, CD31 FL1, Glial fibrillary acidic protein (GFAP), Gross cystic disease fluid protein (GCDFP-15), HMB-45, Human chorionic gonadotropin (hCG), an immunoglobulin, inhibin, keratin (various types), PTPRC (CD45), lymphocyte marker (various types), MART-1 (Melan-A), Myo D1, muscle-specific actin (MSA), neuron-specific enolase (NSE), a neurofilament, placental alkaline phosphatase (PLAP), prostate-specific antigen, S100 protein, smooth muscle actin (SMA), synaptophysin, thyroglobulin, thyroid transcription factor-1, vimentin, Breast Cancer 1 susceptibility protein (BRCA 1) and/or antibody, and Breast Cancer 2 susceptibility protein (BRCA 2) and/or antibody.

According to certain embodiments, a package insert further comprises instructions for preparing buffers and washing solutions. According to certain embodiments, the kit comprises at least one antibody capable of selectively recognizing and/or interacting with a protein according to the teachings of the present invention. In some embodiments, the kit is an ELISA kit. In other embodiments, the kit is an immunohistochemical kit. According to certain additional embodiments, the kit comprises nucleotide probes or primers capable of selectively hybridizing to a polynucleotide according to the teachings of the invention. In yet additional embodiments, the kit is for performing a NAT-based assay. According to some embodiments, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence, In Situ Hybridization or Comparative Genomic Hybridization.

The kits of the present invention can be used inter-alia, for detecting and/or prognosing cancer, detecting precancerous lesions, and monitoring cancer progression.

According to certain aspects of the invention the phrase: “an antibody specifically recognizing a protein according to the teachings of the present invention”, for example “VDAC1 protein antibody” refers to an antibody that specifically and selectively recognize and/or interact with the protein of the invention, e.g. VDAC1, the later also defined herein as “cancer cell marker”.

According to certain embodiments, a cancer cell marker probe is a labeled antibody which specifically recognizes a cancer cell marker. In other embodiments, a cancer cell marker probe is a primary antibody which specifically recognizes a cancer cell marker and a secondary antibody comprising a label. In additional embodiments, a cancer cell marker probe is a labeled nucleic acid molecule which specifically recognizes a cancer cell marker. In another embodiment, a cancer cell marker probe is a labeled protein which specifically recognizes a cancer cell marker. In another embodiment, a cancer cell marker probe is a labeled small molecule which specifically recognizes a cancer cell marker.

In certain embodiments, the label is a dye. In some embodiments, the label is a fluorescent dye. In other embodiments, the label is a radioactive molecule. In yet other embodiments, the label is a metal such as but not limited to gold or silver.

In other embodiments, determining a level of a protein of the invention is quantifying the amount of the protein in a sample by an indirect method such as but not limited to ELISA. In certain embodiments, determining a level of a protein of the invention is performing immunohistochemical analysis on a target tissue and quantifying the intensity and/or number of cells labeled. In other embodiments, any method, for detecting and directly/indirectly quantifying a protein within cells or a tissue, can be applied according to the methods of the invention. In other embodiments, a predetermined reference value is obtained by measuring the level of a protein (or proteins) of the invention in a parallel healthy tissue or cells. In other embodiments, a predetermined reference value is obtained by measuring the level of a protein (or proteins) of the invention in a parallel non-malignant tissue or cells. In other embodiments, a predetermined reference value is obtained by measuring the level of a protein (or proteins) of the invention in a parallel inflamed tissue.

In this context, examining a biological sample for evidence of dysregulated cell growth (such as overexpression VDAC1 and/or MAVS and/or other cancer cells markers according to the teachings of the present invention in cancers and in precancerous lesions) allows for early detection of such aberrant physiology, before a pathologic state such as cancer has progressed to a stage that therapeutic options are more limited and or the prognosis is worse. In such examinations, the status of the proteins of the present invention, for example VDAC1 and/or MAVS expression in a biological sample of interest can be compared, in one embodiment, to the status of VDAC1 and/or MAVS expression in a corresponding non-malignant sample or normal sample (e.g. a sample from that individual or alternatively another individual that is not affected by cancer or any other pathology). In some embodiments, an alteration in the status of VDAC1 and/or MAVS expression in the biological sample (as compared to the normal sample) provides evidence of dysregulated cellular growth. In addition to using a biological sample that is not affected by a pathology as a normal sample or a non-malignant sample, one can also use a predetermined normative value such as a predetermined normal level of mRNA expression (see, e.g., Grever et al., J. Comp. Neurol. 1996. 376(2): 306-14 and U.S. Pat. No. 5,837,501) to compare VDAC1 and/or MAVS status in a sample.

According to certain embodiments, a sample according to the invention comprises a tissue or cells isolated from a subject. According to other embodiment, a sample according to the invention comprises isolated peripheral blood lymphocytes (PBLs) of a subject in need thereof. The level of protein or mRNA can be measured in the sample as was obtained from the subject or the sample can be processed before the protein and/or mRNA level is detected as is known to a person skilled in the art.

According to some embodiment, the phrase “peripheral blood cell” refers to a sample taken from circulating blood as opposed to blood cells sequestered within the lymphatic system, spleen, liver, or bone marrow. In certain embodiments, the term refers to large granular lymphocytes and small lymphocytes. In other embodiments, large granular lymphocytes include natural killer cells (NK cells). Small lymphocytes consist of T cells and B cells.

According to certain embodiment, the term “isolated” refers to isolated from the natural environment. According to typical embodiments, the term relates to blood or tissue sample isolated from a subject to be diagnosed. According to certain embodiment, peripheral blood cell samples are typically taken using a syringe with a needle. Methods of processing peripheral blood cell samples are known in the art and further described in the Examples section herein below. According to some embodiments, the peripheral blood samples are processed to separate the blood serum. According to other embodiments, tissue samples are taken employing methods known to a person skilled in the art.

According to certain embodiments, cancers or pre-cancer lesions that can be diagnosed according to the invention include but are not limited to: melanoma, intraocular melanoma, neoplasms of the central nervous system, tumors of the gastrointestinal tract (colon cancer, rectum cancer, anal region cancer, colorectal cancer, small and/or large bowel cancer, esophageal cancer, stomach cancer, pancreatic cancer, gastric cancer, small intestine cancer, adenocarcinoma arising in the small intestine, carcinoid tumors arising in the small intestine, lymphoma arising in the small intestine, mesenchymal tumors arising in the small intestine, gastrointestinal stromal tumors), gallbladder carcinoma, Biliary tract tumors, prostate cancer, kidney (renal) cancer (e.g., Wilms' tumor), liver cancer (e.g., hepatoblastoma, hepatocellular carcinoma), hepatobiliary cancer, biliary tree cancer, tumors of the Gallbladder, bladder cancer, embryonal rhabdomyosarcoma, germ cell tumor, trophoblastic tumor, testicular germ cells tumor, immature teratoma of ovary, epithelial ovarian, sacrococcygeal tumor, choriocarcinoma, placental site trophoblastic tumor, epithelial adult tumor, ovarian cancer, cervical cancer, cancer of the vagina, cancer of the Vulva, lung cancer (e.g., small-cell and non-small cell lung carcinoma), nasopharyngeal, breast cancer, squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic, cutaneous T-cell lymphoma, primary central nervous system lymphoma), gliomas, medullary thyroid carcinoma, testicular cancer, brain and head/neck cancer, gynecologic cancer, endometrial cancer, germ cell tumors, mesenchymal tumors, neurogenic tumors, cancer of the bladder, cancer of the ureter, cancer of the renal pelvis, cancer of the urethra, cancer of the penis, cancers of the uterine body, uterine sarcoma, peritoneal carcinoma and Fallopian Tube carcinoma, germ cell tumors of the ovary, sex cord-stromal tumors, cancer of the endocrine system, thyroid tumors, medullary thyroid carcinoma, thyroid lymphoma, parathyroid tumors, adrenal tumors, sarcomas of the soft tissue and bone, malignant mesothelioma, skin cancer, cutaneous medulloblastomas, meningiomas, peripheral nerve tumors, Pineal region tumors, pituitary adenomas, craniopharyngiomas, acoustic neuromas, Glomus Jugulare tumors, Chordomas and Chondrosarcomas, Hemangioblastomas, Choroid Plexus Papillomas and Carcinomas, and spinal axis tumors.

According to certain typical embodiments, a pre-cancer lesion is diagnosed in a non-solid tumor. According to some embodiment, a pre-cancer lesion is diagnosed in a hematological malignancy.

In other embodiments, the methods of the invention are practiced on a subject in need thereof. As used herein the phrase “subject in need thereof” refers to a human subject who is at risk of having cancer e.g., a genetically predisposed subject, a subject with medical and/or family history of cancer, a subject who has been exposed to carcinogens, occupational hazard, environmental hazard and/or a subject who exhibits suspicious clinical signs of cancer e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness), changes to the shape or color of existing moles, itching, bleeding or ulcerating moles. Additionally or alternatively, the subject in need thereof can be a healthy human subject undergoing a routine well-being check. According to further embodiment, a “subject in need thereof” refers to a human subject who has cancer.

As used herein, the term “level” refers to the degree of gene expression and/or gene product expression or activity in the biological sample. Accordingly, the level of a protein of the invention serving as a marker is determined, in some embodiments, at the amino acid level using protein detection methods.

In some embodiments, the level of a protein of the invention is determined using a specific antibody via the formation of an immunocomplex (i.e., a complex formed between the protein-antigen present in the biological sample and the specific antibody). In certain embodiments, the immunocomplex of the present invention can be formed at a variety of temperatures, salt concentration and pH values which may vary depending on the method and the biological sample used. Those of skills in the art are capable of adjusting the conditions suitable for the formation of each immunocomplex.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. (Proc. Nat'l Acad. Sci. USA 1972, 69, 2659-2662). Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (scFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are described, for example, by Whitlow and Filpula (Methods 1991 2, 97-105); Bird et al., 1988 Science 242, 423-426; Pack et al., 1993, Bio/Technology 11, 1271-77); and U.S. Pat. No. 4,946,778.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry (Methods 1991 2, 106-110).

Antibodies can also be produced using various techniques known in the art, including phage display libraries (Hoogenboom and Winter, J. Mol. Biol., 1991 227, 381; Marks et al., J. Mol. Biol., 1991 222:581). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 1991 147(1), 86-95). Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 1992 10, 779-783); Lonberg et al., Nature 1994 368, 856-859); Morrison, Nature 1994 368, 812-813); Fishwild et al., Nature Biotechnology 1996 14, 845-51); Neuberger, Nature Biotechnology 1996 14, 826); and Lonberg and Huszar, 1995 Intern. Rev. Immunol. 13, 65-93).

According to the method of this aspect of the present invention, an amount of immunocomplex formation is indicative of a diagnosis or prognosis of the cancer. Various methods can be used to detect the formation of the marker-protein immunocomplex of the present invention and those of skills in the art are capable of determining which method is suitable for each immunocomplex. Antibodies for the protein array used according to the teachings of the present invention are known in the art.

In another embodiment, the immunocomplex of the present invention is labeled using methods known in the art. It will be appreciated that the labeled antibodies can be either primary antibodies (i.e., which bind to the specific antigen) or secondary antibodies (e.g., labeled goat anti rabbit antibodies, labeled mouse anti human antibody) which bind to the primary antibodies. The antibody can be directly conjugated to a label or can be conjugated to an enzyme.

In another embodiment, the antibodies of the present invention are fluorescently labeled (using a fluorescent dye conjugated to an antibody), radiolabeled (using radiolabeled e.g., ¹²⁵I, antibodies), or conjugated to an enzyme (e.g., horseradish peroxidase or alkaline phosphatase) and used along with a chromogenic substrate to produce a colorimetric reaction. The chromogenic substrates utilized by the enzyme-conjugated antibodies of the present invention include, but are not limited to, AEC, Fast red, ELF-97 substrate [2-(5′-chloro-2-phosphoryloxyphenyl)-6-chloro-4(3H)-quinazolinone], p-nitrophenyl phosphate (PNPP), phenolphthalein diphosphate, and ELF 39-phosphate, BCIP/INT, Vector Red (VR), salmon and magenta phosphate (Avivi C., et al., 1994, J Histochem. Cytochem. 1994 42, 551-4) for alkaline phosphatase enzyme and Nova Red, diaminobenzidine (DAB), Vector(R) SG substrate, luminol-based chemiluminescent substrate for the peroxidase enzyme. These enzymatic substrates are commercially available from Sigma (St Louis, Mo., USA), Molecular Probes Inc. (Eugene, Oreg., USA), Vector Laboratories Inc. (Burlingame, Calif., USA), Zymed Laboratories Inc. (San Francisco, Calif., USA), Dako Cytomation (Denmark).

In another embodiment, detection of the immunocomplex is performed using fluorescence activated cell sorting (FACS), enzyme linked immunosorbent assay (ELISA), Western blot and radio-immunoassay (RIA) analyses, immunoprecipitation (IP) with optionally the use of magnetic beads or by a molecular weight-based approach.

In another embodiment, detection of the marker is performed by immunoprecipitation (IP). For immunoprecipitation analysis the marker antibody directly interacts with a sample (e.g., cell lysate) and the formed complex can be further detected using a secondary antibody conjugated to beads. The beads can be then precipitated by centrifugation, following which the precipitated proteins can be detached from the beads (e.g., using denaturation at 95° C.) and further subjected to Western blot analysis using the specific antibodies. Alternatively, in another embodiment, the anti-marker antibody and the beads-conjugated secondary antibody may be added to the biological sample containing the antigen to thereby form an immunocomplex, followed by Western blot analysis with anti-marker antibodies.

In other embodiments, the expression profiles of MAVS and/or VDAC1 and/or additional proteins according to the teachings of the present invention make these proteins diagnostic markers for metastasized disease. In certain embodiments, the expression levels of VDAC1 and/or MAVS and/or SMAC/DIABLO and/or AIF and/or Bcl-2 and/or HK-1 gene products provide information useful for predicting a variety of factors including susceptibility to advanced stage disease, rate of progression, and/or tumor aggressiveness.

In other embodiments, the methods of the present invention comprise measuring the level of mRMA encoding the protein cancer markers of the invention.

The polynucleotide levels in a patient sample can be analyzed by a variety of protocols that are well known in the art, including hybridization and/or nucleic acid technology (NAT)-based assays. RNA detection methods are performed using an isolated polynucleotide (e.g., a polynucleotide probe, an oligonucleotide probe/primer) capable of hybridizing to a marker nucleic acid sequence such as the VDAC1 transcript.

Such a polynucleotide can be at any size, such as a short polynucleotide (e.g., of 15-200 bases), an intermediate polynucleotide of 100-2000 bases and a long polynucleotide of more than 2000 bases. In some embodiments, an isolated polynucleotide probe used by the present invention can be any directly or indirectly labeled RNA molecule (e.g., RNA oligonucleotide (e.g., of 17-50 bases), an in vitro transcribed RNA molecule), DNA molecule (e.g., oligonucleotide, e.g., 15-50 bases, cDNA molecule, genomic molecule) and/or an analogue thereof (e.g., peptide nucleic acid (PNA)) which is specific to the marker RNA transcript of the present invention. In another embodiment, oligonucleotides designed according to the teachings of the present invention can be generated according to any oligonucleotide synthesis method known in the art such as enzymatic synthesis or solid phase synthesis

The isolated polynucleotide used according to the teachings of the present invention can be labeled either directly or indirectly using a tag or label molecule. Such labels can be, for example, fluorescent molecules (e.g., fluorescein or Texas Red), radioactive molecule and chromogenic substrates (e.g., Fast Red, BCIP/INT, available, for example, from ABCAM, Cambridge, Mass.). In another embodiment, direct labeling can be achieved by covalently conjugating a label molecule to the polynucleotide (e.g., using solid-phase synthesis) or by incorporation via polymerization (e.g., using an in vitro transcription reaction or random-primed labeling). Indirect labeling can be achieved by covalently conjugating or incorporating to the polynucleotide a non-labeled tag molecule (e.g., Digoxigenin or biotin) and subsequently subjecting the polynucleotide to a labeled molecule (e.g., anti-Digoxigenin antibody or streptavidin) capable of specifically recognizing the non-labeled tag.

The NAT-based assay can be selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence, In Situ Hybridization or Comparative Genomic Hybridization.

Analysis of the expression of the proteins of the invention is also useful, in one embodiment, as a tool for identifying and evaluating agents that modulate the protein activity of their gene expression. In certain embodiments, identification of a molecule or biological agent that inhibits the genes expression or over-expression in cancer cells is of therapeutic value. In another embodiment, such an agent can be identified by using a screen that quantifies the genes expression by RT-PCR, nucleic acid hybridization or antibody binding.

In other embodiments, the present invention can also assess the change in status of the expression of the cancer cell markers of the present invention. The term “status” in this context is used according to its art accepted meaning and refers to the condition or state of a gene and, particularly, its products including mRNA and protein. Typically, skilled artisans use a number of parameters to evaluate the condition or state of a gene and its products. These include, in some embodiments, but are not limited to the location of expressed gene products (including the location of the marker expressing cells) as well as the level, and biological activity of expressed gene products (such as mRNA and polypeptides). In another embodiment, an alteration in the status of the cancer cell markers mRNA or protein comprises a change in the location of the mRNA or protein and/or the cancer cell marker expressing cells and/or an increase in the cancer cell marker mRNA and/or protein expression, or any combination thereof.

The cancer cell marker status (expression profile, quantity-level, location, etc.) in a sample can be analyzed by a number of means well known in the art, including without limitation, immunohistochemical analysis, in situ hybridization, RT-PCR analysis on laser capture micro-dissected samples, Western blot analysis, and tissue array analysis. In another embodiment, protocols for evaluating the status of a cancel cell marker gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Thus, the status of the cancer cell markers according to the teachings of the present invention in a biological sample is evaluated by various methods utilized by skilled artisans including, but not limited to Northern analysis and/or PCR analysis (to examine, for example alterations in the polynucleotide sequences or expression levels), and, Western and/or immunohistochemical analysis (to examine, for example alterations in polypeptide sequences, alterations in polypeptide localization within a sample, alterations in expression levels and/or associations of the proteins with polypeptide binding partners).

In another embodiment, the expression profile of the proteins and mRNAs according to the teachings of the present invention makes them act as diagnostic markers for local and/or metastasized disease, and provides information on the growth or oncogenic potential of a biological sample. In particular, the status of the cancer cell markers provide information useful for predicting susceptibility to particular disease stages, progression, and/or tumor aggressiveness.

In another embodiment, the status of the cancer cell markers of the invention in a biological sample is examined by a number of well-known procedures in the art. For example, the status of the cancer cell markers in a biological sample taken from a specific location in the body can be examined by evaluating the sample for the presence or absence of cells expressing the markers. In another embodiment, examination can provide evidence of dysregulated cellular growth, for example, when cells-expressing the markers are found in a biological sample that does not normally contain such cells, because such alterations in the status of the mRNA and/or proteins of the invention in a biological sample are often associated with dysregulated cellular growth. In another embodiment, one indicator of dysregulated cellular growth is the metastases of cancer cells from an organ of origin to a different area of the body.

A further aspect of the invention is an assessment of the susceptibility that an individual has for developing cancer. In one embodiment, a method for predicting susceptibility to cancer comprises detecting VDAC1 and/or MAVS mRNA or VDAC1 and/or MAVS protein in a tissue sample, its presence indicating susceptibility to cancer, wherein the degree of VDAC1 and/or MAVS mRNA expression correlates to the degree of susceptibility. In another embodiment, the presence of VDAC1 and/or MAVS in a non-solid tumor or other tissue is examined, with the presence of VDAC1 and/or MAVS in the sample providing an indication of cancer susceptibility. Cross-analysis of the VDAC1 and/or MAVS expression levels with relevant clinical data available (including, for example, pathology grade and tumor-nodes-metastasis (TNM) staging) can be used to produce a database for evaluating the potential of VDAC1 and/or MAVS in combination with associated proteins as is known in the art and as disclosed herein to serve as predictive biomarker for disease state and treatment efficacy.

According to certain embodiments, the assessment of the susceptibility of the subject for developing cancer further comprises detecting at least one additional protein selected from the group consisting of, but not limited to, SAMC/DIABLO, AUF, HK-1 and Bcl-2.

In another embodiment, the invention also comprises methods for gauging tumor aggressiveness. In one embodiment, a method for gauging aggressiveness of a tumor comprises determining the level of VDAC1 and/or MAVS mRNA or VDAC1 and/or MAVS protein expressed by tumor cells, comparing the level so determined to the level of VDAC1 and/or MAVS mRNA or VDAC1 and/or MAVS protein expressed in a corresponding normal tissue taken from the same individual or a normal tissue reference sample, wherein the degree of VDAC1 and/or MAVS mRNA or VDAC1 and/or MAVS protein expression in the tumor sample relative to the normal sample indicates the degree of aggressiveness. In another embodiment, aggressiveness of a tumor is evaluated by determining the extent to which VDAC1 and/or MAVS is expressed in the tumor cells, with higher expression levels indicating more aggressive tumors.

According to certain embodiments, the gauging tumor aggressiveness further comprises determining the level of at least one additional protein selected from the group consisting of, but not limited to, SAMC/DIABLO, AUF, HK-1 and Bcl-2.

In another embodiment, diagnostic approaches are combined with any one of a wide variety of prognostic and diagnostic protocols known in the art. In another embodiment, the invention is also directed to methods for observing a coincidence between the expression of the cancer cell markers of the invention and a factor that is associated with malignancy, as a means for diagnosing and prognosticating the status of a tissue sample. In another embodiment, a wide variety of factors associated with malignancy can be utilized, such as the expression of genes associated with malignancy as well as gross cytological observations (see, e.g., Bocking et al., 1984, Anal. Quant. Cytol. 6(2):74-88; Epstein, 1995, Hum. Pathol. 26(2):223-9; Thorson et al., 1998, Mod. Pathol. 11(6):543-51; Baisden et al., 1999, Am. J. Surg. Pathol. 23(8):918-24).

In another embodiment, diagnostic compositions of the present invention comprise an article of manufacture e.g., kit, such as an FDA approved kit, which contains diagnostic reagents and instructions for use. The kit, in some embodiments, is accommodated by a notice associated with the container in a form prescribed by a governmental agency regulating the manufacture, use or sale of pharmaceuticals, which notice is reflective of approval by the agency of the form of the compositions or human or veterinary use.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996). Other general references are provided throughout this document.

### Material and Methods

### Materials

Phenylmethylsulfonyl fluoride (PMSF), propidium iodide (PI), and trypan blue were purchased from Sigma (St. Louis, Mo.). Dulbecco's modified Eagle's medium (DMEM) and the supplements, fetal calf serum, L-glutamine and penicillin-streptomycin, were purchased from Biological Industries (Beit Haemek, Israel). Anti-Bcl-2 and anti-VDAC1 mouse monoclonal antibodies were obtained from Calbiochem (Nottingham, UK); the polyclonal antibodies were obtained from Abcam (Cambridge, UK). Anti-actin monoclonal antibodies were from Millipore (Billerica, Mass.). Goat polyclonal anti-HK-1 (N19) and anti-HK-II (C14) antibodies were obtained from Santa Cruz Biotechnology (Santa Cruz, Calif.). Monoclonal Bax (YTH-2D2) antibodies were obtained from Travigen (Gaithersburg, Md.). Rabbit polyclonal anti-MAVS antibody, monoclonal anti-AIF antibody (E20, ab32516) and rabbit polyclonal anti-SMAC/DIABLO antibody (ab8115) were from Abcam (Cambridge, UK). Monoclonal anti-CD5/CD19 antibodies were obtained from BD Bioscience (San Jose, Calif.). Horseradish peroxidase (HRP)-conjugated anti-mouse, anti-rabbit and anti-goat antibodies were from Promega.

### Profiling of Protein Expression in Non-Solid Tumors

### Patients

Forty patients with chronic lymphoid leukemia (CLL) from Soroka University Medical Center, including 23 women and 17 men with a median age of 66 years were included in the study. Diagnosis of CLL was based on clinical examination, peripheral blood count and immuno-phenotyping. Patients were not receiving any treatment for the disease. 64% of the patients were at stage 0-1, 20% at stage 2 and 16% at stage 3-4.

The research proposal was approved by the Advisory Committee on Ethics in Human Experimentation.

### Isolation of Peripheral Blood Mononuclear Cells (PBMCs)

PBMCs were isolated from venous blood of the participating subjects by Ficoll-Paque PLUS (GE Healthcare, Israel) density gradient centrifugation, as follows: after informed consent, venous blood (10-20 ml) was drawn from CLL patients with satisfying diagnostic criteria for CLL or from normal adult donors. Blood was collected into heparin tubes and was diluted 1:1 with balance solution composed of two stock solutions, solution A (1% D-glucose, 50 mM CaCl₂, 0.98 mM MgCl₂, 5.4 mM KCl and 0.145 M Tris-HCl, pH 7.6) and solution B (0.14 M NaCl) in a 1:9 ratio, respectively. The resulting mix was carefully layered on Ficoll-Paque Plus (10 ml of diluted blood:15 ml Ficoll) in 50 ml conical tubes. The tubes were centrifuged at 400×g (with minimal acceleration and deceleration) and 18-20° C. for 40 min. The fine layer of mononuclear cells was transferred to a new centrifuge tube, washed 3 times with balance solution and resuspended in culture medium appropriate to the application.

PBMCs were also maintained up to one week in DMEM supplemented with 10% fetal calf serum, 2 mM L-glutamine, 100 U/ml penicillin, 100 μg/ml streptomycin, 1 mM sodium pyruvate, non-essential amino acids, 10 mM Hepes and 11 μM β-mercaptoethanol (all from Biological Industries, Israel) in a humidified chamber of 95% air, 5% CO₂ at 37°. Fractions of the PBMCs were cryo-preserved in liquid nitrogen in 90% FCS, 10% DMSO at concentration of 2.5×10⁷ cells/ml. After thawing, the PBMCs were maintained as described above. No significant differences in the results obtained from assays conducted using fresh cells, tissue culture-maintained cells or frozen cells were noted.

The proportion of cancerous B cells out of the total PBMC pool was analyzed by detection of CD19/CD5 double positive cells using specific antibodies and using a flow cytometer (Beckton-Dickinson, San Jose, Calif.).

### Gel Electrophoresis and Immunoblotting

PBMCs of CLL patients and healthy donors were incubated for 15 minutes on ice with lysis buffer (50 mM Tris-HCl, pH 7.5, 150 mM NaCl, 1 mM EDTA, 1.5 mM MgCl₂, 10% glycerol, 1% Triton X-100, supplemented with a protease inhibitor cocktail (Calbiochem)). Cell lysates were then centrifuged at 17,500×g (15 min at 4° C.) and samples (10-40 mg of protein) were subjected to SDS-PAGE. Gels were stained with Commassie Brilliant Blue or electro-transferred onto nitrocellulose membranes for immunostaining. Membranes containing the transferred proteins were blocked with 5% non-fat dry milk and 0.1% Tween-20 in Tris-buffered saline and incubated overnight at 4° C. with the different primary antibodies, followed by incubation with the relevant HRP-conjugated secondary antibody for 1 h. After treatment with the appropriate primary and secondary antibodies, blots were developed using enhanced chemiluminescence (Biological Industries). Band intensities was analyzed by densitometry using Multi Gauge software (Fujifilm) and the values were normalized to the intensities of the appropriate -actin signal that was used as a loading control.

### Mitochondrial Mass

Cells were loaded with 20 to 50 nM Mitotracker Green (Molecular Probes) for 30 min at 37° C. The probe is taken up into mitochondria, producing a fluorescence signal which is not affected by mitochondrial membrane potential, thereby providing a measure of mitochondrial mass.

### Real-time PCR

Total RNA from PBMCs of 10 CLL patients and 6 healthy donors was isolated using the RNEasy Mini kit (Qiagen, Valencia, Calif.) as described by the manufacturer. Complementary DNA was synthesized from 1 μg total RNA using the High Capacity cDNA Reverse Transcription Kit with RNase Inhibitor and random hexamer primers (Applied Biosystems, Foster city, CA). RT-PCR was performed in triplicates of 20 μl reaction volumes using TaqMan master mix and TaqMan specific probes and primers for HK-1 (Hs00175976_m1), HK-II (Hs01034061_g1) and VDAC1 (Hs01631624_gH (Applied Biosystems, Foster city, CA). The expression levels of the target genes were normalized relative to GAPDH (Hs99999905_m1) levels. Samples were amplified in a 7500 Real Time PCR System (Applied Biosystems, Foster City, Calif.) for 40 cycles using the following PCR parameters: 95° C. for 30 seconds, 57° C. for 1 minute, and 72° C. for 1 minute. The mean fold changes plus or minus SEM of the 3 replicates were calculated.

### Statistics

All the descriptive statistics for data analysis were computed using the statistical package SPSS, version 17.0. Bivarience analysis was performed using a binary logistic regression model with a 95% confidence interval. The independent variable was the relative protein expression level determined by immunostaining, as described above. Means±SEM of results obtained from the indicated independent experiments are presented. The level of significance of difference between the control (healthy) and experimental (CLL patients) groups was determined by Student's test. P values of <0.01 (**) or <0.001 (***) were considered significant.

### Immunohistochemistry Analysis

The expression level of the apoptotic and metabolism-related proteins according to the teachings of the present invention was analyzed using tissue microarray (TMA) slides and immunehistochemistry. TMA slides were purchased from US-Biomax (http://www.biomax.us/index.php). For each cancer type 30 samples from different subjects having malignancy and 7 control samples from different normal subjects were obtained. A broad range of tumor types was examined, including, melanoma, brain, breast, cervix, colon, esophagus, head and neck, kidney, liver, lymph nodes, ovary, pancreas, prostate, stomach, testis, thyroid, uterine and lung cancers.

Formalin-fixed/paraffin-embedded tissue sections were deparaffinized in xylene, and rehydrated in a series of decreasing ethanol/H₂O solutions. Antigens were retrieved by steam heat for 20 min in a sodium citrate buffer (10 mM sodium citrate, 0.05% Tween 20, pH 6.0), followed by endogenous peroxidase blocking in 2.5% H₂O₂ in PBS for 2 min. Subsequently, the slides were blocked in 10% normal serum, 1% BSA in PBS 0.1% Triton X-100 for 2 hours at room temperature. Tissues were incubated with VDAC1 primary antibody (Abcam, ab15895-1:1000) or with MAVS primary antibody (1:500) in 5% gout serum, 1% BSA in PBS overnight at 4° C. The slides were then washed three times with PBS 0.1% Triton, followed by incubation with HRP-conjugated secondary antibody diluted in PBS 1% BSA for 2 hours at room temperature. For HRP detection the sections were developed in 3,3′-diaminobenzidine (DAB; Vector Labs, Burlingame Calif., USA) chromogen according to the manufacture recommendations, followed by counterstaining with hematoxylin (Sigma-Aldrich). Images were captured using a Nikon inverted microscope (Nikon Instruments Inc. Melville N.Y., USA). The differences in expression levels between cancer and normal tissues was quantified using the Ariol platform (Leica Microsystems, Germany) and/or by experienced pathologists of Soroka University Medical centre, Beer Sheva.

### Example 1

### MAVS is a Valid Marker for Non-Solid Tumors

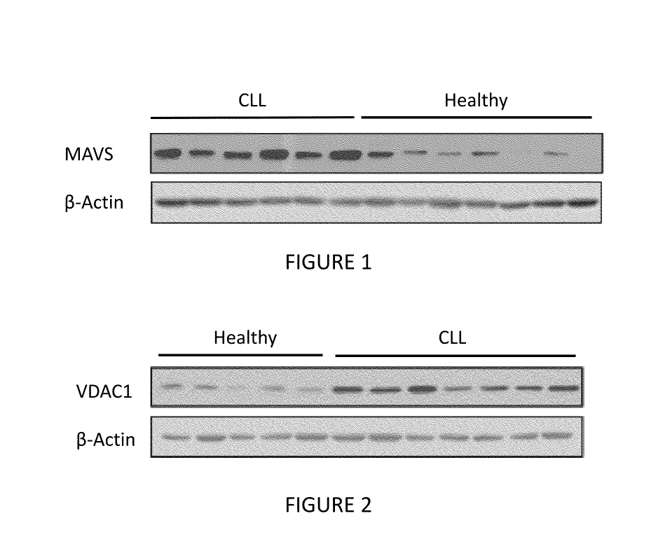

Peripheral blood mononuclear cells (PBMC) were isolated from: (1) a group of patients afflicted with chronic lymphocytic lymphoma; and (2) healthy donors group. The expression levels of β-actin and MAVS were measured. As shown in FIG. 1, MAVS is highly expressed (4.6 average fold expression) in blood cells isolated from chronic lymphocytic lymphoma patients compared to blood cells isolated from healthy volunteers. Therefore, MAVS can serve as a marker for chronic lymphocytic lymphoma.

### Example 2

### VDAC1 is a Valid Marker for Non-Solid Tumors

Peripheral blood mononuclear cells (PBMC) were isolated from: (1) a group of patients afflicted with chronic lymphocytic lymphoma (CLL); and (2) healthy donors group. The expression level of VDAC1 was measured, and normalized to the expression level of β-actin. As shown in FIG. 2, VDAC1 is highly expressed (6.4 fold average expression) in blood cells isolated from chronic lymphocytic lymphoma patients compared to blood cells isolated from healthy volunteers. Therefore, VDAC1 can serve as marker/markers for chronic lymphocytic lymphoma.

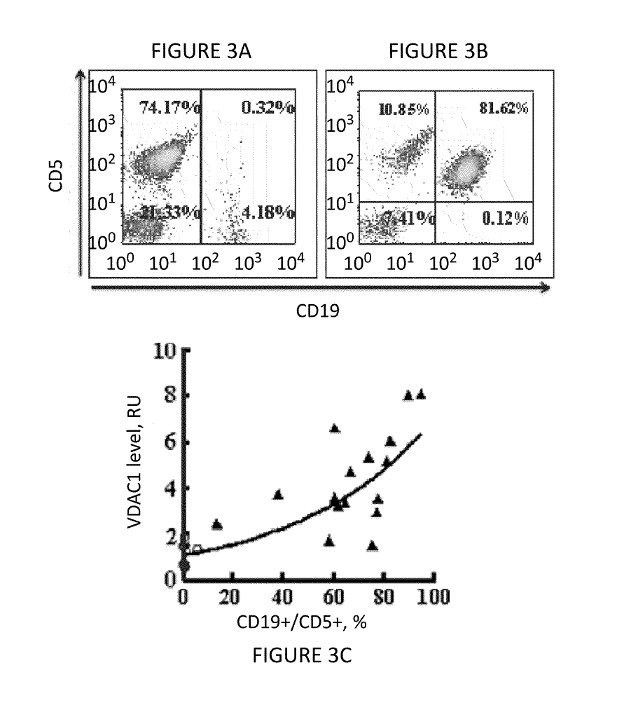

The relationship between VDAC1 expression level and the relative amounts of CD5+/CD19+ cells was examined. Cells expressing CD5+/CD19+ represent the CLL cancerous cells that are present in PBMCs isolated from each CLL patient. Representative flow cytometry analysis of PBMCs from a single CLL patient and a healthy donor demonstrates that in the CLL patient (FIG. 3B), about 82% of the cells express CD19+/CD5+, while less than 1% of such cells are found in PBMCs from a healthy donor (FIG. 3A).

The level of the VDAC1 expression was plotted as a function of the percentage of CD5+/CD19+ cells for each patient (FIG. 3C). The results revealed that the level of VDAC1 is positively correlated with the amount of CD5+/CD19+ cells. This correlation further demonstrates that VDAC1 expression level is strongly associated with cancerous CLL cells.

### Example 3

### Apoptosis and Metabolism-Related Proteins as Markers for Non-Solid Tumors

The expression level of several proteins related to mitochondrial metabolism and apoptosis was examined in peripheral blood mononuclear cells (PBMCs) obtained from patient afflicted with chronic lymphocytic lymphoma (CLL) and from healthy donors, using antibodies specific to each of the proteins. The protein examined, in addition to VDAC1 and MAVS described above, were Bcl-2, apoptosis inducing factor (AIF), second mitochondrial activator of caspases/direct IAP-binding protein with low isoelectric point (SMAC/DIABLO) and hexokinase-1 (HK-1).

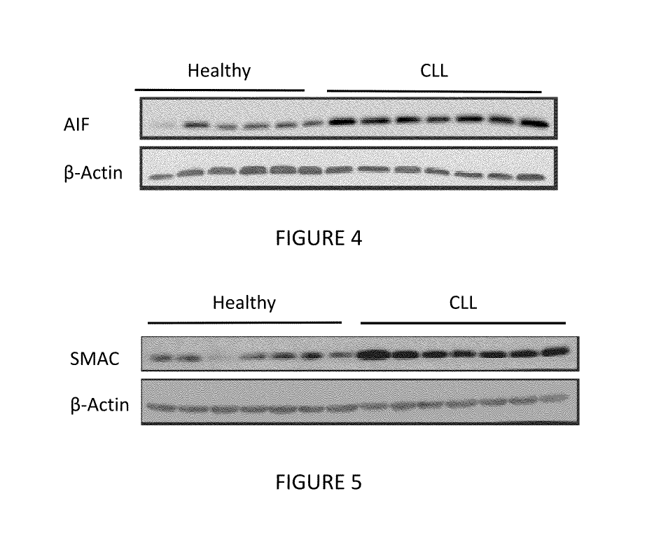

AIF and SMAC/DIABLO were over-expressed by an average of 3.0 and 3.9 fold, respectively, in PBMCs derived from CLL patients compared to PBMCs derived from healthy donors (FIG. 4 and FIG. 5, respectively).

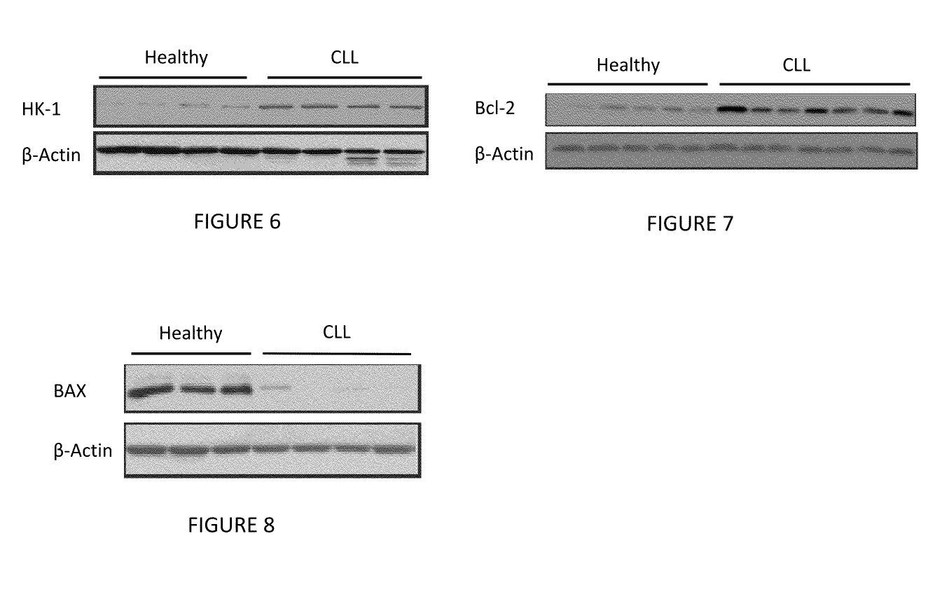

To assess HK levels, several commercially available antibodies directed against HK-1 or HK-II, most of which display non-isoform-specific reactivity, were used. In PBMCs isolated from CLL patients HK-1 expression was slightly increased (1.35-fold, FIG. 6) compared to cells isolated from healthy subjects, as was, to lesser extent, HK-II expression (data not shown),

A comparison of the expression level of the anti-apoptotic protein Bcl-2 and of the pro-apoptotic protein Bax in PBMCs derived from CLL and healthy donors showed that Bcl-2 protein levels are markedly higher in PBMCs obtained from CLL patients (FIG. 7) while Bax protein levels are significantly lower (FIG. 8). Intensity analysis of the Western blots revealed that Bcl-2 expression was 9.4-fold higher in CLL, while Bax expression reached a level of less than 10% of the expression measured in cells of healthy donors. The expression levels of the mitochondrial translocator protein (TSPO), the pro-apoptotic protein Bak and lactate dehydrogenase (LDH) were also examined and found to remain unchanged or to show only a slight difference in PBMCs isolated from CLL compared to those isolated from healthy individuals (data not shown).

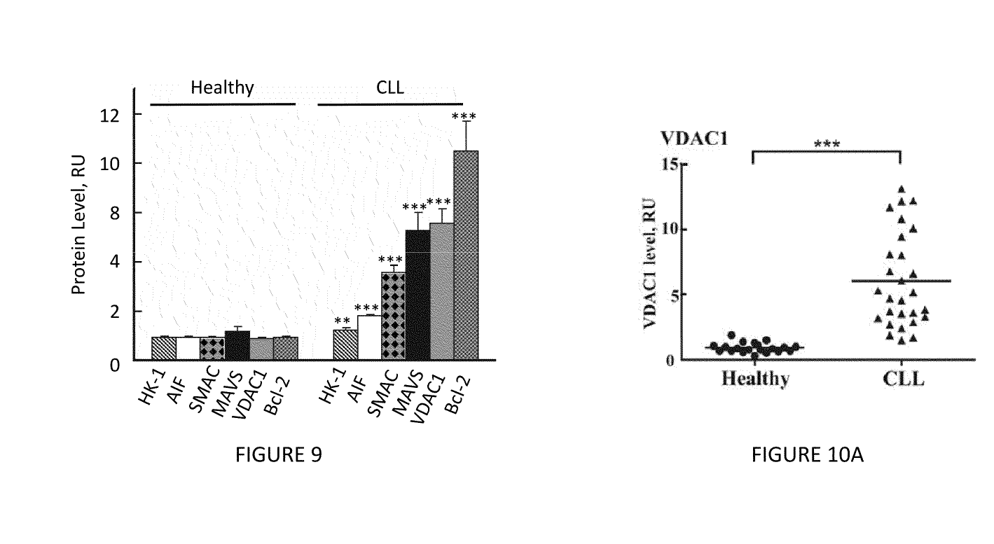

The above-described results are summarized in FIG. 9, showing the expression level of the proteins normalized to the expression level of β-actin. The means (±SD) of three independent Western blots for each sample are presented (**, p=0.01, ***p=0.001). To verify whether the increase in the protein expression is the result of an increase in mitochondrial numbers/mass, PBMCs derived from CLL and healthy donors were stained with MitoTracker green, a dye that is localized to mitochondria regardless of mitochondrial membrane potential. FACS analysis of cell stained with MitoTracker green revealed similar staining in healthy and CLL derived PBMCs (data not shown).

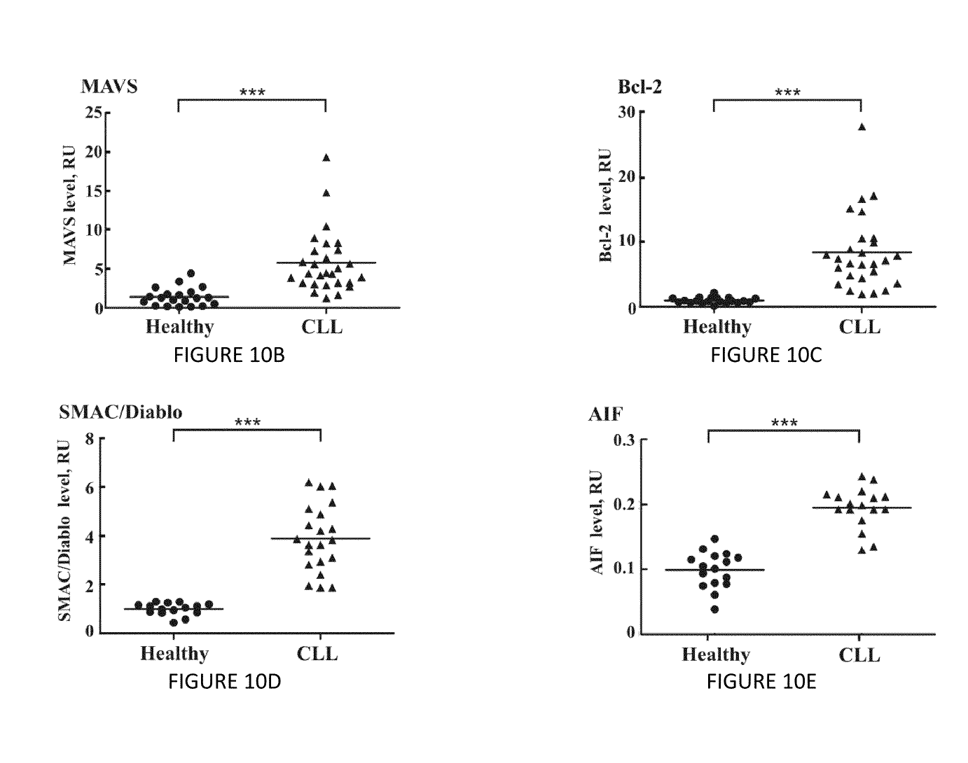

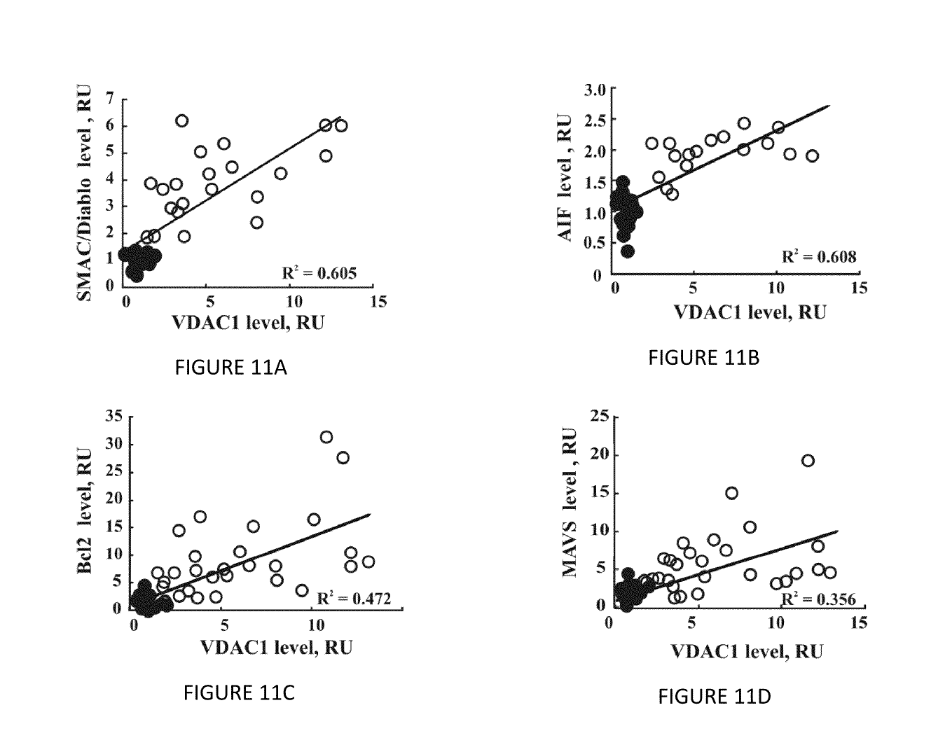

The results for the expression level of MAVS, VDAC1, SAMC/DIABLO, AIF and Bcl-2 in PBMCs isolated from CLL patients and healthy subjects are also presented as vertical scattered plots showing significant differences between the two groups in each case (FIG. 10). About 80% of the CLL patients show VDAC1, Bcl2, MAVS and SMAC/Diablo expression levels higher than the expression levels observed in all healthy donors. In some CLL patients, Bcl2 and VDAC1 expression levels were up to 20- and 14-fold higher than in the healthy donors, respectively. Similarly, SMAC/Diablo and MAVS were over-expressed in some CLL patients up to 6- and 20-fold higher than in the healthy donors, respectively. The relationship between VDAC1 expression level and that of the other apoptosis modulator proteins over-expressed in CLL was then analyzed (FIG. 11). VDAC1 over-expression is positively correlated with the expression levels of SMAC/Diablo (FIG. 11A, R²=0.6052), AIF (FIG. 11B, R²=0.6077), Bcl-2 (FIG. 11C, R²=0.4715) and MAVS (FIG. 11D, R²=0.356). HK-1 expression was weakly correlated with that of VDAC1, as the HK-1 expression level was only slightly increased in CLL. These results suggest that the expression level of VDAC1, known to play a key role in regulating the cell energy and thus cell metabolism in addition to its role in cell apoptosis, is associated with the expression of the other apoptosis modulator proteins described herein.

### Example 4

### A Protein Array as Marker for Predicting Non-Solid Tumors

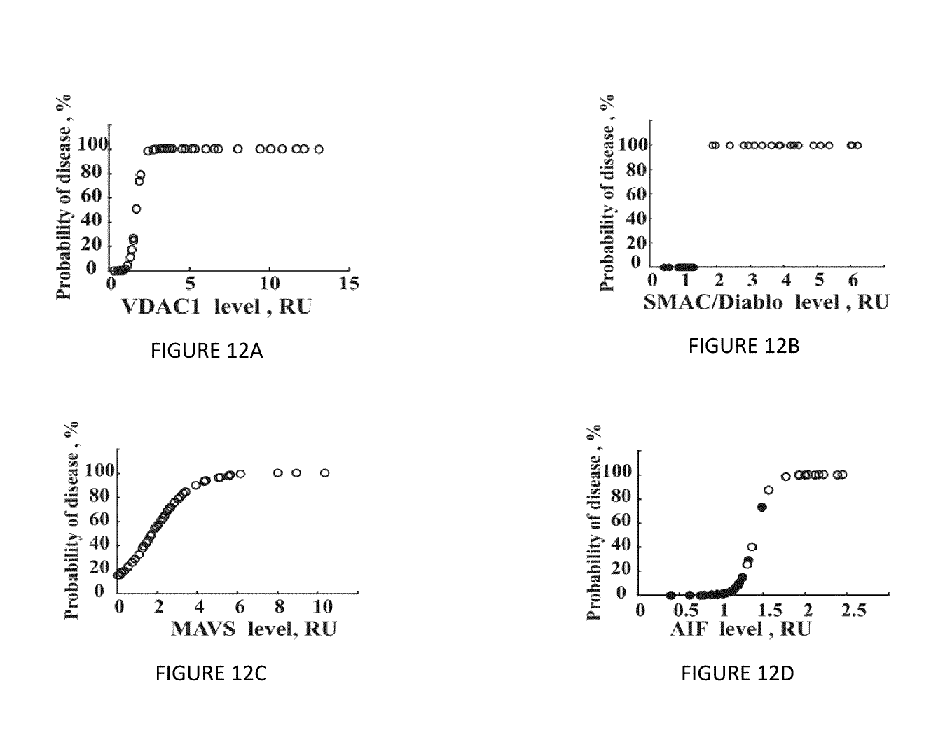

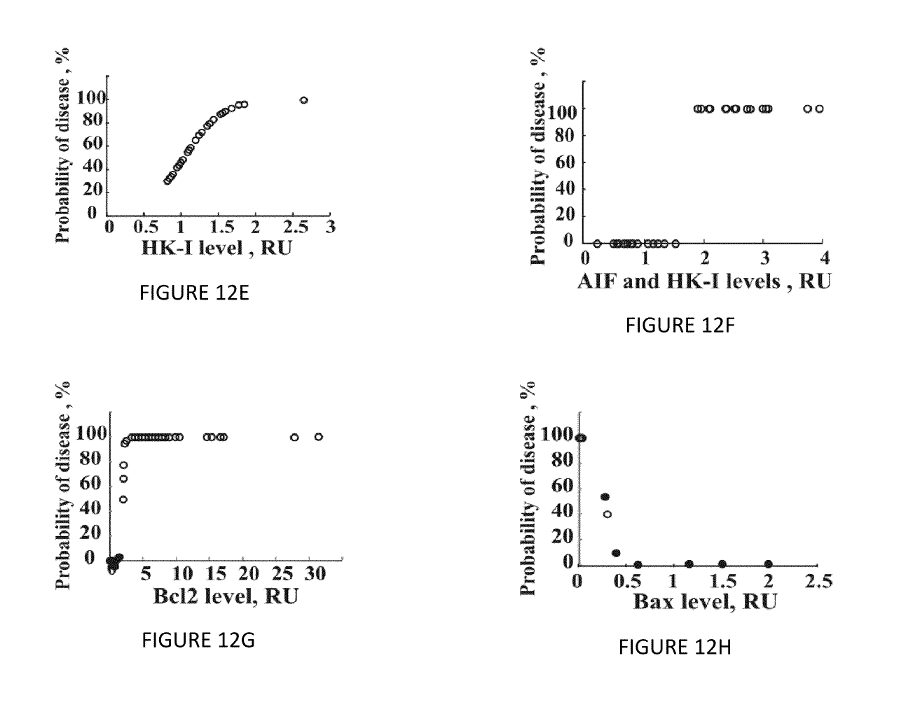

A binary logistic regression model was employed to examine the ability to predict, according to the expression level of each of the apoptosis modulator proteins, whether a sample was obtained from a healthy subject or from a CLL patient. In this analysis, the relationship between the probability of disease (0 to 100) and the expression level of the analyzed protein was determined for each apoptosis modulator protein. The binary logistic regression model was carried out with a 95% confidence interval (FIG. 12).

For VDAC1, Bcl2, SMAC/Diablo and MAVS, the ability to predict occurrence of the disease based on expression level is over 95% (Table 1). On the other hand, predication of disease based on the HK-1 expression level is weaker. This, however, improved to 100% when the data of HK-1 was combined with AIF (FIG. 12F) or with another apoptosis modulator (data not shown). This analysis clearly demonstrates the possibility to determine disease occurrence based on the expression levels of one or a combination of several of the apoptotic modulator proteins analyzed herein.

TABLE 1
Expression of apoptosis modulator proteins in CLL: Specificity,
sensitivity and overall disease predication

| Protein | Specificity % | Sensitivity % | Predictive value, % |
|---|---|---|---|
| SMAC/Diablo | 100 | 100 | 100 |
| VDAC1 | 95 | 96.4 | 95.8 |
| Bcl-2 | 95 | 96.4 | 95.8 |
| AIF | 93.8 | 88.2 | 90.9 |
| BAX | 83.3 | 83.3 | 83.3 |
| MAVS | 80 | 89.3 | 85.4 |
| HK-II | 90 | 76.9 | 82.6 |
| HK-1 | 52.6 | 67.9 | 61.7 |
| AIF × HK-1 | 100 | 100 | 100 |

Sensitivity (predicted CLL patients/total CLL patients) and the specificity (predicted healthy donors/total healthy donors) as CLL biomarkers were determined for the apoptotic-regulating proteins analyzed based on a cut-off value of 0.5 using binary logisitic regression analysis. The predictive values represent the averages of the sensitivity and specificity values.

### Example 5

### VDAC1 Expression Level Correlates with Tumor Progression

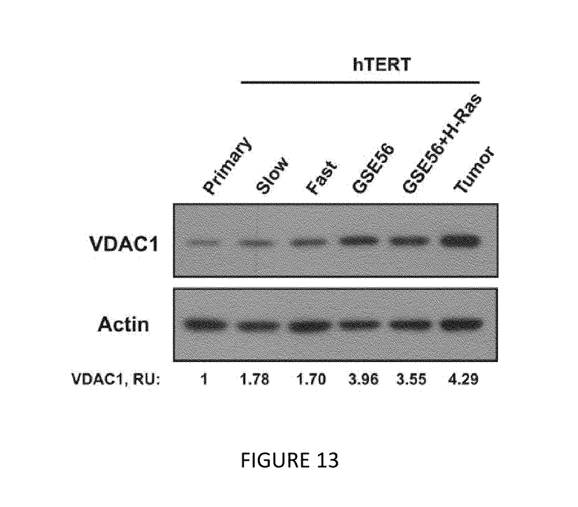

The cancer cell markers VDAC1 not only provides a tool for assessing the presence of a tumor but also provides an important tracking system for determining tumor progression stage (staging tumor progression). According to the conclusive results provided in FIG. 13 the expression levels of the cancer cell marker VDAC1 intensifies as the tumor progresses.

### Example 6

### MAVS and VDAC1 are Valid Markers for Solid Tumors

The expression level of the proteins MAVS and VDAC1 was also examined in a vast number of solid tumors. Analysis was performed using tissue microarray (TMA) slides (US-Biomax). 30 different malignant tissue samples and 7 control tissue samples were analyzed for each cancer type. Immunohistochemistry analysis for the presence of MAVS and VDAC was performed as described in the material and method section herein above.

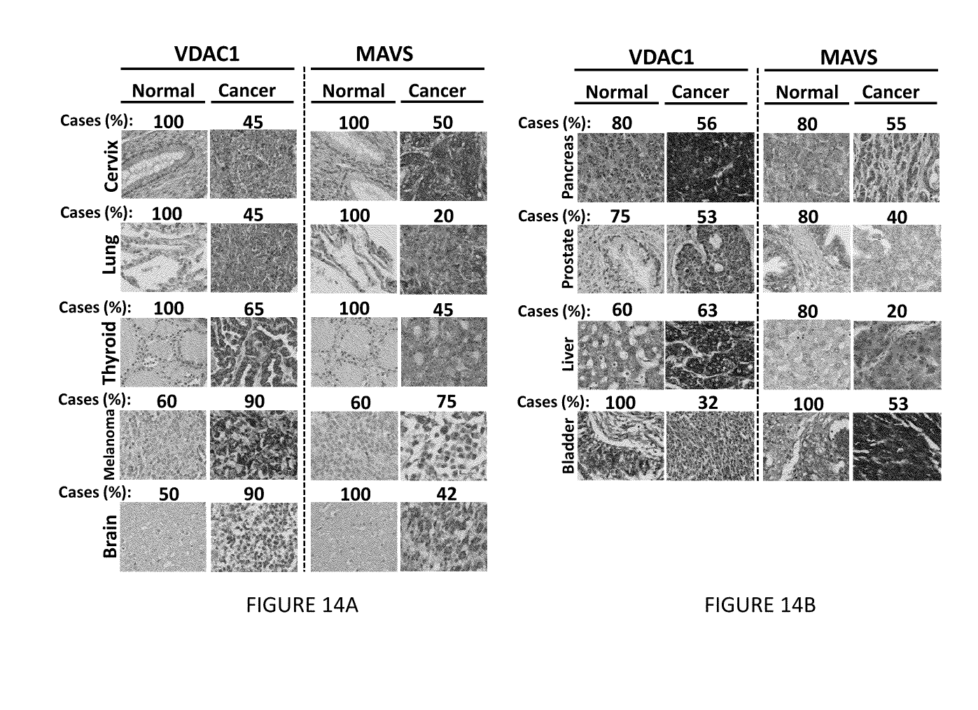

Table 2 summarizes the expression level of MAVS and VDAC1 in each of the malignant samples examined compared to the control samples. Selected stained slide are presented in FIG. 14A-B.

TABLE 2
VDAC1 and MAVS expression levels in different types of cancer

| | VDAC1 | | MAVS | |
|---|---|---|---|---|
| Tissue | Cancer | Normal | Cancer | Normal |
| Melanoma | +++ | + | ++ | +(Nevus tissue) |
| Thyroid | +++ | − | + | − |
| Breast | +++ | ++/+++ | ++ | ++ |
| Lung | +++ | − | ++/+++ | − |
| Lymph node | ++/+++ | +/++ | Not Detected | Not Detected |
| Bladder | +++ | ++ | +++ | ++ |
| Prostate | +++/+ | +/− | ++/+ | ++ |
| Brain | ++/+++ | +/++ | ++/+++ | − |
| Ovary | ++/+++ | + | ++ | + |
| Stomach | +++ | +++ | ++ | ++ |
| Liver | +++ | +/++ | + | +/− |
| Testis | +++/++ | +/++ | ++/+ | ++/+ |
| Colon | +++ | +++ | +++ | +++ |
| Pancreas | +++/++ | + | ++ | +/− |
| Uterus | +++ | +++ | +++ | ++ |
| Kidney | +++ | ++ | ++ | + |
| Cervix uteri | +++ | − | +++ | − |
| Esophagus | +++ | + | Not Detected | Not Detected |
| Head and neck | +++/++ | Not Detected | Not Detected | Not Detected |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for diagnosing cancer in a subject, the method comprising the steps of:
(a) determining a level of a mitochondrial antiviral-signaling (MAVS) protein (GeneBank accession Nos. NP_{—}001193420.1 and NP_{—}065797.2) or mRNA in a sample obtained from the subject; and
(b) comparing the level of the MAVS protein or mRNA to a predetermined threshold;
wherein MAVS level above the predetermined threshold is indicative for the presence of cancer cells in the subject, thereby diagnosing cancer in said subject.

2. A method for diagnosing cancer in a subject, the method comprising the steps of:
(a) determining a level of voltage-dependent anion channel 1 (VDAC1) protein (GeneBank accession Nos. ABM87491.1, ABM84109.1 and AAH90042.1) or mRNA in a sample obtained from the subject; and
(b) comparing the level of the VDAC1 protein or mRNA to a predetermined threshold;
wherein VDAC1 level above the predetermined threshold is indicative for the presence of cancer cells in the subject, thereby diagnosing cancer in said subject.

3. A method for diagnosing cancer in a subject, the method comprising the steps of
(a) determining in a sample obtained from the subject a level of voltage-dependent anion channel 1 (VDAC1) protein (GeneBank accession Nos. ABM87491.1, ABM84109.1 and AAH90042.1) or mRNA and a level of at least one additional cancer marker selected from the group consisting of:
(i) mitochondrial antiviral-signaling (MAVS, GeneBank accession Nos. NP_{—}001193420.1 and NP_{—}065797.2) protein or mRNA;
(ii) second mitochondria-derived activator of caspases (SMAC/DIABLO, UniProt accession No. Q9NR28) protein or mRNA;
(iii) apoptosis inducing factor (AIF, UniProt accession No. 095831) protein or mRNA;
(iv) Bcl-2 (UniPort accession No. P10415.2) protein or mRNA; and
(v) hexokinase-1 (HK-1, GeneBank accession No. AAC15862.1 protein or mRNA; and any combination thereof; and
(b) comparing the level of the VDAC1 protein or mRNA and the level of the at least one additional cancer marker to a predetermined threshold;
wherein level above the predetermined threshold of said VDAC1 protein or mRNA and of said at least one additional protein or mRNA is indicative for the presence of cancer cells in the subject, thereby diagnosing cancer in said subject.

4. The method of claim 3, further comprising determining the level of VDAC1 and MAVS, wherein level above a predetermined threshold of VDAC1 and MAVS protein or mRNA is indicative for the presence of cancer cells in the subject, thereby diagnosing cancer in said subject.

5. The method of claim 3, further comprising determining the level of VDAC1 and SMAC/DIABLO, wherein a level above a predetermined threshold of VDAC1 and SMAC/DIABLO protein or mRNA is indicative for the presence of cancer cells in the subject, thereby diagnosing cancer in said subject.

6. The method of claim 3, further comprising determining the level of VDAC1, HK-1 and at least one additional protein or mRNA selected from the group consisting of MAVS, SMAC/DIABLO and AIF, wherein level above a predetermined threshold of VDAC1, HK-1 and the at least one additional protein or mRNA is indicative for the presence of cancer cells in the subject, thereby diagnosing cancer in said subject.

7. The method of claim 3, further comprising determining the level of VDAC1, Bcl-2 and at least one additional protein or mRNA selected from the group consisting of MAVS, SMAC/DIABLO and AIF, wherein level above a predetermined threshold of VDAC1, Bcl-2 and the at least one additional protein or mRNA is indicative for the presence of cancer cells in the subject, thereby diagnosing cancer in said subject.

8. The method of claim 3, further comprising determining the level of VDAC1, MAVS, SMAC/DIABLO and AIF, wherein a level above a predetermined threshold of VDAC1, MAVS, SMAC/DIABLO and AIF protein or mRNA is indicative for the presence of cancer cells in the subject, thereby diagnosing cancer in said subject.

9. The method of claim 3, further comprising determining the level of VDAC1, MAVS, SMAC/DIABLO, AIF, Bcl-2 and HK-1, wherein a level above a predetermined threshold of VDAC1, MAVS, SMAC/DIABLO, AIF, Bcl-2 and HK-1 protein or mRNA is indicative for the presence of cancer cells in the subject, thereby diagnosing cancer in said subject.

10. The method of claim 1, wherein the cancer is a non-solid tumor or a solid tumor.

11. The method of claim 2, wherein the cancer is a non-solid tumor or a solid tumor.

12. The method of claim 3, wherein the cancer is a non-solid tumor or a solid tumor.

13. A method for monitoring tumor progression in a subject, the method comprising the steps of:
(a) determining a level of voltage-dependent anion channel 1 (VDAC1) protein (GeneBank accession Nos. ABM87491.1, ABM84109.1 and AAH90042.1) or mRNA in a sample comprising tumor cells isolated from the subject; and
(b) comparing the level of the VDAC1 protein or mRMA to a predetermined threshold; wherein VDAC1 level above the predetermined threshold correlates with tumor progression, thereby monitoring tumor progression.

14. The method of claim 13, further comprising the steps of:
determining in the sample the level of at least one additional protein or mRNA selected from the group consisting of Mitochondrial antiviral-signaling (MAVS, GeneBank accession Nos. NP_{—}001193420.1 and NP_{—}065797.2); Second mitochondria-derived activator of caspases (SMAC/DIABLO, UniProt accession No. Q9NR28; Apoptosis inducing factor (AIF, UniProt accession No. 095831); Bcl-2 (UniPort accession No. P10415.2); Hexokinase-1 (HK-1, GeneBank accession No. AAC15862.1); and any combination thereof; and
comparing the level of the at least one additional protein or mRNSA to a predetermined threshold; wherein level above the predetermined threshold of VDAC1 and said at least one additional protein or mRNA correlates with tumor progression, thereby monitoring tumor progression.

15. The method of claim 14, wherein the tumor is a non-solid tumor or a solid tumor.

16. A method for monitoring tumor progression in a subject, comprising the steps of:
(a) determining a level of Mitochondrial antiviral-signaling (MAVS) protein GeneBank accession Nos. NP_{—}001193420.1 and NP_{—}065797.2) or mRNA in a sample comprising tumor cells isolated from the subject; and
(b) comparing the level of the MAVS protein or mRNA to a predetermined threshold;
wherein MAVS level above the predetermined threshold correlates with tumor progression, thereby monitoring tumor progression.

17. The method of claim 16, wherein the tumor is a non-solid tumor or a solid tumor.

18. A kit for the diagnosis or prognosis of cancer, the kit comprising:
(a) at least two probes for detecting (i) a voltage-dependent anion channel 1 (VDAC1) protein or mRNA and (ii) at least one additional cancer cell marker selected from the group consisting of mitochondrial antiviral-signaling (MAVS, GeneBank accession Nos. NP_{—}001193420.1 and NP_{—}065797.2) protein or mRNA; second mitochondria-derived activator of caspases (SMAC/DIABLO, UniProt accession No. Q9NR28) protein or mRNA; apoptosis inducing factor (AIF, UniProt accession No. 095831) protein or mRNA; Bcl-2 (UniPort accession No. P10415.2) protein or mRNA; hexokinase-1 (HK-1, GeneBank accession No. AAC15862.1) protein or mRNA or any combination thereof; and
(b) a package insert comprising instructions for measuring the level of the VDAC1 protein or mRNA and the at least one additional cancer cell marker protein or mRNA.

19. The kit of claim 18, further comprising a nucleotide probe or primer capable of selectively hybridizing to VDAC1 mRNA and at least one additional probe or primer capable of selectively hybridizing to a mRNA of at least one of MAVS, SMAC/BIABLU, AIF, Bcl-2 and HK-1.

20. The kit of claim 18, further comprising an antibody capable of selectively recognizing or interacting with VDAC1 protein and at least one additional antibody capable of selectively recognizing or interacting with at least one protein selected from the group consisting of MAVS, SMAC/DIABLO, AIF, Bcl-2 and HK-1.
